(19) United States
(12) Patent Application Publication
(10) Pub. No.: US 2009/0286823 A1
(43) Pub. Date: Nov. 19, 2009

(54) CCR1 Inhibitors useful for the treatment of multiple myeloma and other disorders

(75) Inventors: Ole Petter Veiby, Westborough, MA (US); Kenneth C. Anderson, Wellesley, MA (US); Sonia Vallet, Boston, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals Inc., Cambridge, MA (US)

(21) Appl. No.: 12/316,901

(22) Filed: Dec. 17, 2008

Related U.S. Application Data
(60) Provisional application No. 61/007,943, filed on Dec. 17, 2007.

(51) Int. Cl. A61K 31/4545; A61P 35/00

(52) U.S. Cl. 514/291

(57) ABSTRACT

The invention relates to the use of inhibitors of CCR1 for the treatment of cancers and osteolytic bone disorders. In some embodiments, the invention relates to methods for the treatment of multiple myeloma, smoldering multiple myeloma and secondary bone cancers.

This application claims priority from U.S. Provisional Patent Application No. 61/007,943, filed Dec. 17, 2007, which is hereby incorporated by reference in its entirety.

### BACKGROUND OF THE INVENTION

Multiple myeloma (MM) is a B-cell malignancy of the plasma cells. Multiple myeloma results from the clonal proliferation of these plasma cells arising in the lymph nodes and “homing” to the bone marrow where these cells localize and proliferate. Proliferation of the cancerous plasma cells, referred to as myeloma cells, causes a variety of effects, including lytic lesions (holes) in the bone, decreased red blood cell number, production of abnormal proteins (with attendant damage to the kidney, nerves, and other organs), reduced immune system function, and elevated blood calcium levels (hypercalcemia).

Although responsible for a small number of all cancers in the United States, with about 15,980 new cases expected to be diagnosed in 2005, myeloma is the second most common blood cancer (to non-Hodgkins) and may be increasing in prevalence, particularly among individuals under age 55 (American Cancer Society and International Myeloma Foundation). This trend toward more frequent myeloma in patients under 55 implies that environmental causative factors in the past 60 years may be playing a significant role (American Cancer Society and International Myeloma Foundation). Currently, many different treatment options are available or in development, but there is no cure for multiple myeloma. Patients are treated with chemotherapy as well as symptom-specific treatments for one or more of hypercalcemia, increased infection risk, bone destruction and pain, and muscle weakness (see International Myeloma Foundation, Concise Review of the Disease and Treatment Options, 2005 Ed.). Even with successful management of the disease, there remains a risk of remission of the disease that may be resistant to current therapies.

Monoclonal gammopathy of undetermined significance (MGUS) and smoldering multiple myeloma (SMM) are asymptomatic, pre-malignant disorders characterized by monoclonal plasma cell proliferation in the bone marrow and absence of end-organ damage.

Smoldering multiple myeloma (SMM) is an asymptomatic proliferative disorder of plasma cells with a high risk of progression to symptomatic, or active multiple myeloma (N. Engl. J. Med. 356(25): 2582-2590 (2007)). International consensus criteria defining SMM were adopted in 2003 and require that a patient have a M-protein level of ≧30 g/L and/or bone marrow clonal plasma cells ≧10% (Br. J. Haematol. 121: 749-57 (2003)). The patient must have no organ or related tissue impairment, including bone lesions or symptoms (Br. J. Haematol. 121: 749-57 (2003)). Recent studies have identified two subsets of SMM; i) patients with evolving. disease and ii) patients with non-evolving disease (Br. J. Haematol. 121: 631-636 (2003)). International consensus criteria defining MGUS require that a patient have a M-protein level of <30 g/L, bone marrow plasma cells <10% and the absence of organ or related tissue impairment, including bone lesions or symptoms (Br. J. Haematol. 121: 749-57 (2003)).

SMM resembles monoclonal gammopathy of undetermined significance (MGUS) as end-organ damage is absent (N. Engl. J. Med. 356(25): 2582-2590 (2007)). Clinically, however, SMM is far more likely to progress to active multiple myeloma or amyloidosis at 20 years (78% probability for SMM vs. 21% for MGUS) (N. Engl. J. Med. 356(25): 2582-2590 (2007)).

One of the most prevalent and significant characteristics of myeloma is the activation of osteoclasts; multinucleated cells that absorb bone, leading to bone thinning, lytic bone lesions, and bone fracture. Lytic bone lesions occur in 70-80% of multiple myeloma patients and are frequently associated with severe bone pain and pathologic fractures. In normal bone functioning, a balance exists between osteoclasts, which resorb bone, and osteoblasts, cells that produce bone. This balance is upset in myeloma patients, and more bone is resorbed than produced. The increased osteoclastic bone resorption occurs adjacent to the myeloma cells and not in areas of normal bone marrow, indicating that the osteoclast activation occurs by a local mechanism. It has been shown that myeloma cells, in culture, produce or induce production of several osteoclast-activating factors (OAFs). One of these osteoclast-activating factors has been identified as the chemokine macrophage inflammatory protein-1α (MIP-1α) (Blood 96: 671-675 (2000)), and it has been shown that multiple myeloma cells express MIP-1α (Br. J. Haematol. 120: 53-55; Blood 100: 2195-2202 (2002); Blood 101: 3778-3783 (2003)). Several studies have also correlated MIP-1α expression with osteolytic bone disease in multiple myeloma patients (Br. J. Haematol. 123: 106-109 (2003); Blood 101: 4998-5006 (2003); Br. J. Haematol. 120: 53-55 (2003); and Blood 96: 671-675 (2000)). Additionally, several studies have investigated the effects of MIP-1α on osteoclasts. Specifically, it has been shown that MIP-1α induces osteoclast differentiation in human bone marrow cultures (Blood 97: 3349-3353 (2001)), that bone marrow plasma from multiple myeloma patients induce osteoclast formation (Blood 96: 671-675 (2000)), and that multiple myeloma cells co-cultured with rabbit bone cells enhance osteoclast formation and resorption pit formation on bone slides (Blood 100: 2195-2202 (2002).

One of the receptors for MIP-1α is CCR1 which has been shown to be expressed by multiple myeloma cells, osteoclast precursors, and mature osteoclasts (Leukemia 17: 203-210 (2003); J. Bone Miner. Res. 19: 2065-2077 (2002); J. Cell. Biochem. 87: 386-393 (2002); J. Cell. Physiol. 183: 196-207 (2000)). CCR1 ligands have also been shown to induce recruitment, and differentiation of osteoclast precursors (J. Bone Miner. Res. 19: 2065-2077 (2002)). It has also been recently demonstrated that an anti-CCR1 antibody and a CCR1 small molecule antagonist inhibit MIP-1α osteoclast formation in vitro and inhibit myeloma cell adherence to stromal cell and IL-6 production by stromal cells in response to myeloma cella (Exp. Hematol. 33: 272-278 (2005)). Inhibition of MIP-14 production or function has also been shown to inhibit bone lesions, decrease tumor burden, and increase survival in rodent models of multiple myeloma disease (J. Clin. Invest. 108: 1833-1841 (2001); Cancer 97: 813-817 (2003); Blood 102: 311-319 (2003)).

Taken together, these studies suggest that CCR1 antagonists are useful for the treatment of cancer, including multiple myeloma, and for the treatment of other bone disorders resulting from the chemotactic and other responses of osteoclasts to the CC chemokine macrophage inflammatory protein (MIP-1α). Thus, there is a need to identify antagonists of CCR1 that are effective for the treatment of cancer, including multiple myeloma, and other osteolytic bone disorders.

### DESCRIPTION OF THE FIGURES

FIG. 1: Osteoclast formation and TRAP assay in the presence of CCR1 inhibitor.

FIG. 2: Osteoclast activity assayed by pit formation assay in the presence of CCR1 inhibitor.

FIG. 3: Osteoclast and multiple myeloma adhesion assay in the presence of CCR1 inhibitor.

FIG. 4: Long-term co-culture of osteoclasts and multiple myeloma cells in the presence of CCR1 inhibitor.

FIG. 5: Short-term co-culture of osteoclasts and multiple myeloma cells in the presence of CCR1 inhibitor.

### DESCRIPTION OF THE INVENTION

The present invention is based on the observations that CCR1 inhibitors block osteoclast development and function by inhibiting differentiation of osteoclast precursors. Moreover, CCR1 inhibitors abrogate multiple myeloma cell migration and adhesion to osteoclasts, thus preventing the horning of multiple myeloma cells to osteoclast sites. In addition, CCR1 inhibition overcomes the protective effect of osteoclasts on multiple myeloma cell survival and proliferation, thereby inhibiting the interactive loop between osteoclasts and multiple myeloma cells. Furthermore, CCR1 inhibition may also decrease multiple myeloma growth factor production, including IL-6, from the tumor microenvironment. Thus, CCR1 inhibition may be an effective tool to treat multiple myeloma, to treat or to prevent or to delay the progression of monoclonal gammopathy of undetermined significance (MGUS) or smoldering multiple myeloma (SMM) to multiple myeloma (MM). CCR1 inhibition may also be an effective tool to block, and possibly even prevent, osteolytic bone disease in multiple myeloma.

Compounds of the present invention are antagonists of chemokine receptor function, especially CCR1. Accordingly, compounds of the present invention are useful in the treatment of multiple myeloma, both in its active form and during periods of clinical remission. Compounds of the present invention are also useful in the treatment of MGUS. Compounds of the present invention are also useful in the prevention or delay of the progression of MGUS to MM. Compounds of the present invention are also useful in the treatment of SMM. Compounds of the present invention are also useful in the prevention or delay of the progression of SMM to MM. Compounds of the present invention are useful in the treatment of osteolytic bone disorders in multiple myeloma. Compounds of the present invention are useful in the treatment of a secondary bone cancer selected from breast, lung, prostate, kidney, or thyroid cancer.

In one embodiment, the present invention provides a method for the treatment of multiple myeloma comprising administering a compound of formula (I), or a pharmaceutical composition thereof.

In another embodiment, the present invention provides a method for the treatment of SMM comprising administering of a compound of formula (I), or a pharmaceutical composition thereof. In another embodiment, the present invention provides a method for the treatment of MGUS comprising administering of a compound of formula (I), or a pharmaceutical composition thereof. In another embodiment, the present invention provides a method for the prevention, or delay, of the progression of SMM to MM comprising administering of a compound of formula (I), or a pharmaceutical composition thereof. In another embodiment, the present invention provides for a method for the prevention, or delay, of the progression of MGUS to MM comprising administering of a compound of formula (I), or a pharmaceutical composition thereof.

In another embodiment, the present invention provides a method for the treatment of osteolytic bone disorders in multiple myeloma comprising administering of a compound of formula (I), or a pharmaceutical composition thereof. In another embodiment, the present invention provides a method for the treatment of a secondary bone cancer selected from breast, lung, prostate, kidney, or thyroid cancer, comprising administering of a compound of formula (I), or a pharmaceutical composition thereof.

Compounds of general formula (I) are described as follows:

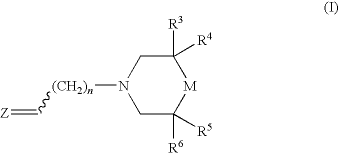

or a pharmaceutically acceptable salt or solvate thereof, or a pharmaceutical composition thereof, wherein:
n is one to four;
M is >CR¹R²;
R¹ is OH or H;
R² is a substituted or unsubstituted aromatic group;
R³ and R⁴ are independently —H, an aliphatic group or a substituted aliphatic group;
R⁵ and R⁶ are independently —H, an aliphatic group or a substituted aliphatic group;
Z is represented by formula (II):

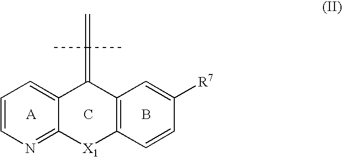

wherein:
ring A is unsubstituted or substituted;
ring B is further unsubstituted or substituted;
R⁷ is —OH, —COOH, —NO₂, halogen, aliphatic group, substituted aliphatic group, an aromatic group, a substituted aromatic group, —NR⁸R⁹, —CONR⁸R⁹, —NR⁸C(O)-(aliphatic group), —NR⁸C(O)-(substituted aliphatic group), —NR⁸S(O)₂-(aliphatic group), —NR⁸S(O)₂-(substituted aliphatic group), —C(O)O-(aliphatic group), —C(O)O-(substituted aliphatic group), —C(O)-(aliphatic group), —C(O)-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹² or —(O)ᵤ—(CH₂)ₜ—NHC(O)O—R¹⁰;
R⁸ and R⁹ are independently —H, an aliphatic group, a substituted aliphatic group, a benzyl group, an aryl group, a non-aromatic heterocyclic group; or R⁸ and R⁹ taken together with the nitrogen atom to which they are bonded can form a substituted or unsubstituted non-aromatic heterocyclic ring;
R¹⁰, R¹¹ or R¹² are independently —H, an aliphatic group, a substituted aliphatic group, an aromatic group, a substituted aromatic group or a non-aromatic heterocyclic group, —NHC(O)—O-(aliphatic group), —NHC(O)—O-(aromatic group) or —NHC(O)—O-(non-aromatic heterocyclic group); or R¹¹ and R¹², taken together with the nitrogen atom to which they are bonded, form a non-aromatic heterocyclic ring;
X₁ is —CH₂—O—;
said aliphatic group is a C₁-C₆ alkyl, alkenyl or alkynyl;
said aromatic group is selected from the group consisting of phenyl, 1-naphthyl, 2-naphthyl, 1-anthracyl, 2-anthracyl, N-imidazolyl, 2-imidazolyl, 4-imidazolyl, 5-imidazolyl, 2-thienyl, 3-thienyl, 2-furanyl, 3-furanyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 4-pyridazinyl, 3-pyrazolyl, 4-pyrazolyl, 5-pyrazolyl, 2-pyrazinyl, 2-thiazolyl, 4-thiazolyl, 5-thiazolyl, 5-tetrazolyl, 2-oxazolyl, 4-oxazolyl, 5-oxazolyl, tetrahydronaphthyl, 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 1-isoquinolinyl, 3-quinolinyl, 1-isoindolyl, 3-isoindolyl, acridinyl, 3-benzisoxazolyl, benzocyclopentyl, benzocyclohexyl;
said non-aromatic heterocyclic group is a five to eight-membered non-aromatic ring which contains one or more heteroatoms independently selected from the group consisting of nitrogen, oxygen or sulfur;
said substituted aliphatic group is substituted with one or more substituents selected from the group consisting of oxo group, epoxy group, non-aromatic heterocyclic ring, benzyl group, substituted benzyl group, aromatic group, substituted aromatic group, electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted non-aromatic heterocyclic group is substituted with one or more substituents selected from the group consisting of ═O, ═S, electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹ R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted aromatic group, substituted benzyl group, Ring A when substituted and Ring B when further substituted, are substituted with one or more substituents selected from the group consisting of electron withdrawing group, halo, azido, —CN, —CONR⁸R⁹, —NR³R⁹, —OS(O)₂NR⁸R⁹, —S(O)₂NR⁸R⁹, —SO₃H, guanidino, oxalo, —C(═NR³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
Q is —O—, —S—, —S(O)—, —S(O)₂—, —OS(O)₂—, —C(O)—, —OC(O)—, —C(O)O—, —C(O)C(O)—O—, —O—C(O)C(O)—, —NHC(O)—, —OC(O)NH—, —NH—C(O)—NH—, —S(O)₂NH—, —NHS(O)₂—, —C(NR¹⁴)NHNH—, —NHNHC(NR¹⁴)—, —NR⁸C(O)— or —NR⁸S(O)₂—;
R¹³ is —H, —OH, —NH₂, an aromatic group or a substituted aromatic group;
R¹⁴ is —H, an aliphatic group, a benzyl group, an aryl group or non-aromatic heterocyclic group;
t is zero to three;
u is zero or one; and
p is one to five.

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

The term “aliphatic” or “aliphatic group”, as used herein, means a substituted or unsubstituted straight-chain, branched or cyclic C₁₋₁₂ hydrocarbon, which is completely saturated or which contains one or more units of unsaturation, but which is not aromatic. For example, suitable aliphatic groups include substituted or unsubstituted linear, branched or cyclic alkyl, alkenyl, alkynyl groups and hybrids thereof, such as (cycloalkyl)alkyl, (cycloalkenyl)alkyl or (cycloalkyl)alkenyl. In various embodiments, the aliphatic group has 1 to 12, 1 to 8, 1 to 6, 1 to 4, or 1 to 3 carbons.

The terms “alkyl”, “alkenyl”, and “alkynyl”, used alone or as part of a larger moiety, refer to a straight and branched chain aliphatic group having from 1 to 12 carbon atoms. For purposes of the present invention, the term “alkyl” will be used when the carbon atom attaching the aliphatic group to the rest of the molecule is a saturated carbon atom. However, an alkyl group may include unsaturation at other carbon atoms. Thus, alkyl groups include, without limitation, methyl, ethyl, propyl, allyl, propargyl, butyl, pentyl, and hexyl.

For purposes of the present invention, the term “alkenyl” will be used when the carbon atom attaching the aliphatic group to the rest of the molecule forms part of a carbon-carbon double bond. Alkenyl groups include, without limitation, vinyl, 1-propenyl, 1-butenyl, 1-pentenyl, and 1-hexenyl.

For purposes of the present invention, the term “alkynyl” will be used when the carbon atom attaching the aliphatic group to the rest of the molecule forms part of a carbon-carbon triple bond. Alkynyl groups include, without limitation, ethynyl, 1-propynyl, 1-butynyl, 1-pentynyl, and 1-hexynyl.

The term “cycloaliphatic”, used alone or as part of a larger moiety, refers to a saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Nonlimiting examples include cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8 members.

In some embodiments, two adjacent substituents on the cycloaliphatic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the term “cycloaliphatic” includes aliphatic rings that are fused to one or more aryl, heteroaryl, or heterocyclyl rings. Nonlimiting examples include indanyl, 5,6,7,8-tetrahydroquinoxalinyl, decahydronaphthyl, or tetrahydronaphthyl, where the radical or point of attachment is on the aliphatic ring. The term “cycloaliphatic” may be used interchangeably with the terms “carbocycle”, “carbocyclyl”, “carbocyclo”, or “carbocyclic”.

The term “aromatic ring” or “aromatic group” refers to “aryl” and all groups included within the term “aryl” and all groups included within the term “heteroaryl” as defined herein.

The terms “aryl” and “ar-”, used alone or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is a C₆₋₁₀ aryl group. Aryl groups include, without limitation, phenyl, naphthyl, and anthracenyl. In some embodiments, two adjacent substituents on the aryl ring, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the term “aryl”, as used herein, includes groups in which an aromatic ring is fused to one or more heteroaryl, cycloaliphatic, or heterocyclyl rings, where the radical or point of attachment is on the aromatic ring. Nonlimiting examples of such fused ring systems include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, fluorenyl, indanyl, phenanthridinyl, tetrahydronaphthyl, indolinyl, phenoxazinyl, benzodioxanyl, and benzodioxolyl. An aryl group may be mono-, bi-, tri-, or polycyclic, preferably mono-, bi-, or tricyclic, more preferably mono- or bicyclic. The term “aryl” may be used interchangeably with the terms “aryl group”, “aryl moiety”, and “aryl ring”.

An “aralkyl” or “arylalkyl” group comprises an aryl group covalently attached to an alkyl group, either of which independently is optionally substituted. Preferably, the aralkyl group is C₆₋₁₀ aryl(C₁₋₆)alkyl, C₆₋₁₀ aryl(C₁₋₄)alkyl, or C₆₋₁₀ aryl(C₁₋₃)alkyl, including, without limitation, benzyl, phenethyl, and naphthylmethyl.

The terms “heteroaryl” and “heteroar-”, used alone or as part of a larger moiety, e.g., heteroaralkyl, or “heteroaralkoxy”, refer to groups having 5 to 14 ring atoms, preferably 5, 6, 9, or 10 ring atoms; having 6, 10, or 14π electrons shared in a cyclic array; and having, in addition to carbon atoms, from one to four heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, two adjacent substituents on the heteroaryl, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono-, bi-, tri-, or polycyclic, preferably mono-, bi-, or tricyclic, more preferably mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclic”, “heterocyclic radical”, and “heterocyclic ring” are used interchangeably and refer to a stable 3- to 7-membered monocyclic, or to a fused 7- to 10-membered or bridged 6- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a heterocyclyl ring having 1-3 heteroatoms selected from oxygen, sulfur or nitrogen, the nitrogen may be N (as in 3,4-dihydro-2H-pyrrolyl), NH (as in pyrrolidinyl) or ⁺NR (as in N-substituted pyrrolidinyl). A heterocyclic ring can be attached to its pendant group at any heteroatom or carbon atom that results in a stable structure, and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

In some embodiments, two adjacent substituents on a heterocyclic ring, taken together with the intervening ring atoms, for an optionally substituted fused 5- to 6-membered aromatic or 3- to 8-membered non-aromatic ring having 0-3 ring heteroatoms selected from the group consisting of O, N, and S. Thus, the terms “heterocycle”, “heterocyclyl”, “heterocyclyl ring”, “heterocyclic group”, “heterocyclic moiety”, and “heterocyclic radical”, are used interchangeably herein, and include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms. The term “partially unsaturated” is intended to encompass rings having multiple sites of unsaturation, but is not intended to include aryl or heteroaryl moieties, as herein defined.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro. Nonlimiting examples of fluoroaliphatics include —CH₂F, —CHF₂, —CF₃, —CH₂CF, —CF₂CH₃, and —CF₂CF₃.

The term “linker group” or “linker” means an organic moiety that connects two parts of a compound. Linkers typically comprise an atom such as oxygen or sulfur, a unit such as —NH—, —CH₂—, —C(O)—, —C(O)NH—, or a chain of atoms, such as an alkylene chain. The molecular mass of a linker is typically in the range of about 14 to 200, preferably in the range of 14 to 96 with a length of up to about six atoms. In some embodiments, the linker is a C₁₋₆ alkylene chain.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

An alkylene chain also can be optionally interrupted by a functional group. An alkylene chain is “interrupted” by a functional group when an internal methylene unit is replaced with the functional group. Examples of suitable “interrupting functional groups” include —C(R*)═C(R*)—, —C≡C—, —O—, —S—, —S(O)—, —S(O)₂—, —S(O)₂N(R⁺)—, —N(R*)—, —N(R⁺)CO—, —N(R⁺)C(O)N(R⁺)—, —N(R⁺)CO₂—, —C(O)N(R⁺)—, —C(O)—, —C(O)—C(O)—, —CO₂—, —OC(O)—, —OC(O)O—, —OC(O)N(R⁺)—, —C(NR⁺)═N, —C(OR*)═N—, —N(R⁺)—N(R⁺)—, or —N(R⁺)S(O)₂—. Each R⁺, independently, is hydrogen or an optionally substituted aliphatic, aryl, heteroaryl, or heterocyclyl group, or two R⁺ on the same nitrogen atom, taken together with the nitrogen atom, form a 5-8 membered aromatic or non-aromatic ring having, in addition to the nitrogen atom, 0-2 ring heteroatoms selected from N, O, and S. Each R* independently is hydrogen or an optionally substituted aliphatic, aryl, heteroaryl, or heterocyclyl group.

Examples of C₃₋₆ alkylene chains that have been “interrupted” with —O-—include —CH₂OCH₂—, —CH₂O(CH₂)₂—, —CH₂O(CH₂)₃—, —CH₂O(CH₂)₄—, —(CH₂)₂OCH₂—, —(CH₂)₂O(CH₂)₂—, —(CH₂)₂O(CH₂)₃—, —(CH₂)₃O(CH₂)—, —(CH₂)₃O(CH₂)₂—, and —(CH₂)₄O(CH₂)—. Other examples of alkylene chains that are “interrupted” with functional groups include —CH₂Z¹CH₂—, —CH₂Z¹(CH₂)₂—, —CH₂Z¹(CH₂)₃—, —CH₂Z¹(CH₂)₄—, —(CH₂)₂Z¹CH₂—, —(CH₂)₂Z¹(CH₂)₂—, —(CH₂)₂Z¹(CH₂)₃—, —(CH₂)₃Z¹(CH₂)—, —(CH₂)₃Z¹(CH₂)₂—, and —(CH₂)₄Z¹(CH₂)—, wherein Z¹ is one of the “interrupting” functional groups listed above.

One of ordinary skill in the art will recognize that when an alkylene chain having an interruption is attached to a functional group, certain combinations are not sufficiently stable for pharmaceutical use. Only stable or chemically feasible compounds are within the scope of the present invention. A stable or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a patient.

The term “substituted”, as used herein, means that a hydrogen radical of the designated moiety is replaced with the radical of a specified substituent, provided that the substitution results in a stable or chemically feasible compound. The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different.

As used herein, the term “independently selected” means that the same or different values may be selected for multiple instances of a given variable in a single compound.

An aryl (including the aryl moiety in aralkyl, aralkoxy, aryloxyalkyl and the like) or heteroaryl (including the heteroaryl moiety in heteroaralkyl and heteroaralkoxy and the like) group may contain one or more substituents. Examples of suitable substituents on the unsaturated carbon atom of an aryl or heteroaryl group include -halo, —NO₂, —CN, —R*, —C(R*)=C(R*)₂, —C≡C—R*, —OR*, —SR^{o}, —S(O)R^{o}, —SO₂R^{o}, —SO₃R^{o}, —SO₂N(R⁺)₂, —N(R⁺)₂, —NR⁺C(O)R*, —NR⁺C(O)N(R⁺)₂, —NR⁺CO₂R—, —O—CO₂R*, —OC(O)N(R⁺)₂, —O—C(O)R*, —CO₂R*, —C(O)—C(O)R*, —C(O)R*, —C(O)N(R⁺)₂, —C(O)N(R⁺)C(═NR⁺)—N(R⁺)₂, —N(R⁺)C(═NR⁺)—N(R⁺)—C(O)R*, —C(═NR)—N(R⁺)₂, —C(═NR⁺)—OR*, —N(R⁺)—N(R⁺)₂, —N(R⁺)C(═NR⁺)—N(R⁺)₂, —NR⁺SO₂R^{o}, —NR⁺SO₂N(R⁺)₂, —P(O)(R*)₂, —P(O)(OR*)₂, —O—P(O)—OR*, and —P(O)(NR⁺)—N(R⁺)₂, wherein R^{o} is an optionally substituted aliphatic or aryl group, and R⁺ and R* are as defined above, or two adjacent substituents, taken together with their intervening atoms, form a 5-6 membered unsaturated or partially unsaturated ring having 0-3 ring atoms selected from the group consisting of N, O, and S.

An aliphatic group or a non-aromatic heterocyclic ring may be substituted with one or more substituents. Examples of suitable substituents on the saturated carbon of an aliphatic group or a non-aromatic heterocyclic ring include, without limitation, those listed above for the unsaturated carbon of an aryl or heteroaryl group and the following: ═O, ═S, ═C(R*)₂, ═N—N(R*)₂, ═N—OR*, ═N—NHC(O)R*, ═N—NHCO₂R^{o}, ═N—NHSO₂R^{o}, or ═N—R*, where each R* and R^{o} is as defined above.

Suitable substituents on the nitrogen atom of a non-aromatic heterocyclic ring include —R*, —N(R*)₂, —C(O)R*, —CO₂R*, —C(O)—C(O)R*, —C(O)CH₂C(O)R*, —SO₂R*, —SO₂N(R*)₂, —C(═S)N(R*)₂, —C(═NH)—N(R*)₂, and —NR*SO₂R*; wherein each R* is as defined above.

Suitable electron withdrawing groups include, for example, alkylimines, alkylsulfonyl, carboxamido, carboxylic alkyl esters, —CH═NH, —CN, —NO₂ and halogens.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to”.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

It also will be apparent to one skilled in the art that certain compounds of this invention may exist in tautomeric forms, all such tautomeric forms of the compounds being within the scope of the invention. Unless stereochemical configuration is expressly defined, structures depicted herein are meant to include all stereochemical forms of the structure; i.e., the R and S configurations for each asymmetric center. Therefore, unless otherwise indicated, single stereochemical isomers as well as enantiomeric and diastereomeric mixtures of the present compounds are within the scope of the invention. Both the R and the S stereochemical isomers, as well as all mixtures thereof, are included within the scope of the invention.

Where stereochemical configuration at a given asymmetric center is defined by structure, unless stated otherwise, the depicted configuration indicates stereochemistry relative to other asymmetric centers in the molecule. Where stereochemical configuration is defined by chemical name, the designations (rel), (R*), and (S*) indicate relative stereochemistry, while the designations (R), (S), (+), (−), and (abs) indicate absolute stereochemistry.

As used herein, the term “diastereomeric purity” refers to the amount of a compound having the depicted relative stereochemistry, expressed as a percentage of the total amount of all diastereomers present. Preferably, the diastereomeric purity of the compound is at least 80%, more preferably at least 90%, still more preferably at least 95%, and most preferably at least 99%.

As used herein, the term “enantiomeric purity” refers to the amount of a compound having the depicted absolute stereochemistry, expressed as a percentage of the total amount of the depicted compound and its enantiomer. Preferably, the enantiomeric purity of the compound is at least 80%, more preferably at least 90%, still more preferably at least 95%, and most preferably at least 99%.

Methods for determining diastereomeric and enantiomeric purity are well-known in the art. Diastereomeric purity can be determined by any analytical method capable of quantitatively distinguishing between a compound and its diastereomers. Examples of suitable analytical methods include, without limitation, nuclear magnetic resonance spectroscopy (NMR), gas chromatography (GC), and high performance liquid chromatography (HPLC). Similarly, enantiomeric purity can be determined by any analytical method capable of quantitatively distinguishing between a compound and its enantiomer. Examples of suitable analytical methods include, without limitation, GC or HPLC, using a chiral column packing material. Enantiomers may also be distinguishable by NMR if first derivatized with an optically enriched derivatizing agent, e.g., Mosher's acid.

The compounds disclosed herein can be obtained as E- and Z-configurational isomers. It is expressly pointed out that the invention includes compounds of the E-configuration and the Z-configuration around the double bond connecting Ring C of Z to the remainder of the molecule, and a method of treating a subject with compounds of the E-configuration, the Z-configuration, and mixtures thereof. Accordingly, in the structural formulas presented herein, the symbol:

[symbol]

is used to represent both the E-configuration and the Z-configuration. Preferably Ring A and the alkylene chain bonded to Ring C are in the cis configuration. For example, the compounds can have the configuration of:

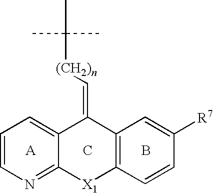

Protecting groups that are suitable for use in the processes and compounds of the present invention are known to those of ordinary skill in the art. The chemical properties of such protecting groups, methods for their introduction and their removal can be found, for example, in T. Greene and P. Wuts, Protective Groups in Organic Synthesis (3^{rd} ed.), John Wiley & Sons, NY (1999).

The chemokine receptor antagonists described herein can be prepared and administered as active compounds or as prodrugs. Generally, prodrugs are analogues of pharmaceutical agents which can undergo chemical conversion by metabolic processes to become fully active. For example, a prodrug of the invention can be prepared by selecting appropriate groups for R⁷. In one embodiment, a prodrug can be represented by compound of formula (V):

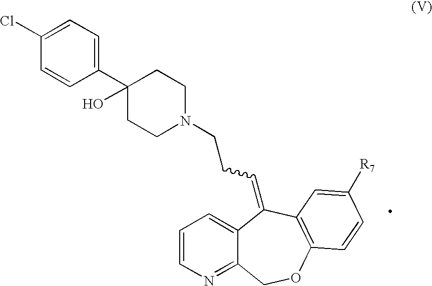

wherein R⁷ is Q-substituted aliphatic group, and the aliphatic group is substituted with —(O)ᵤ—(CH₂)ₜ—C(O)OR¹⁰, wherein Q is —C(O)O—, u is one, t is zero, and R¹⁰ is a cyclic aliphatic group. For example, when the substituted aliphatic group is a substituted ethyl group, R⁷ can be represented by:

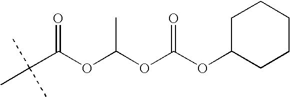

Such a prodrug can be converted to an active chemokine receptor antagonist represented by formula (V), wherein R⁷ is —COOH.

In one embodiment, the method of the invention comprises administering a compound having the following structural formula (I):

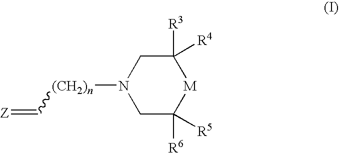

or a pharmaceutically acceptable salt or solvate thereof, or a pharmaceutical composition thereof, wherein:
n is one to four;
M is >CR¹R²;
R¹ is —OH or H;
R² is a substituted or unsubstituted aromatic group;
R³ and R⁴ are independently —H, an aliphatic group or a substituted aliphatic group;
R⁵ and R⁶ are independently —H, an aliphatic group or a substituted aliphatic group;
Z is represented by formula (II):

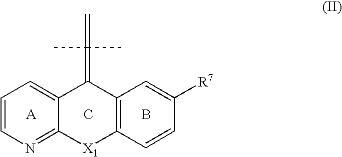

wherein:
ring A is unsubstituted or substituted;
ring B is further unsubstituted or substituted;
R⁷ is —OH, —COOH, —NO₂, halogen, aliphatic group, substituted aliphatic group, an aromatic group, a substituted aromatic group, —NR⁸R⁹, —CONR⁸R⁹, —NR⁸C(O)-(aliphatic group), —NR⁹C(O)-(substituted aliphatic group), —NR⁸S(O)₂-(aliphatic group), —NR⁸S(O)₂-(substituted aliphatic group), —C(O)O-(aliphatic group), —C(O)O-(substituted aliphatic group), —C(O)-(aliphatic group), —C(O)-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹² or —(O)ᵤ—(CH₂)ₜ—NHC(O)O—R¹⁰;
R⁸ and R⁹ are independently —H, an aliphatic group, a substituted aliphatic group, a benzyl group, an aryl group, non-aromatic heterocyclic group; or R⁸ and R⁹ taken together with the nitrogen atom to which they are bonded can form a substituted or unsubstituted non-aromatic heterocyclic ring;
R¹⁰, R¹¹ or R¹² are independently —H, an aliphatic group, a substituted aliphatic group, an aromatic group, a substituted aromatic group or a non-aromatic heterocyclic group, —NHC(O)—O-(aliphatic group), —NHC(O)—O-(aromatic group) or —NHC(O)—O-(non-aromatic heterocyclic group); or R¹¹ and R¹², taken together with the nitrogen atom to which they are bonded, form a non-aromatic heterocyclic ring;
X₁ is —CH₂—O—;
said aliphatic group is a C₁-C₆ alkyl, alkenyl or alkynyl;
said aromatic group is selected from the group consisting of phenyl, 1-naphthyl, 2-naphthyl, 1-anthracyl, 2-anthracyl, N-imidazolyl, 2-imidazolyl, 4-imidazolyl, 5-imidazolyl, 2-thienyl, 3-thienyl, 2-furanyl, 3-furanyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 4-pyridazinyl, 3-pyrazolyl, 4-pyrazolyl, 5-pyrazolyl, 2-pyrazinyl, 2-thiazolyl, 4-thiazolyl, 5-thiazolyl, 5-tetrazolyl, 2-oxazolyl, 4-oxazolyl, 5-oxazolyl, tetrahydronaphthyl, 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 1-isoquinolinyl, 3-quinolinyl, 1-isoindolyl, 3-isoindolyl, acridinyl, 3-benzisoxazolyl, benzocyclopentyl, benzocyclohexyl;
said non-aromatic heterocyclic group is a five to eight-membered non-aromatic ring which contains one or more heteroatoms independently selected from the group consisting of nitrogen, oxygen or sulfur;
said substituted aliphatic group is substituted with one or more substituents selected from the group consisting of oxo group, epoxy group, non-aromatic heterocyclic ring, benzyl group, substituted benzyl group, aromatic group, substituted aromatic group, electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted non-aromatic heterocyclic group is substituted with one or more substituents selected from the group consisting of ═O, ═S, electron withdrawing group, halo, azido, —CN, —CONR⁸R⁹, —NR³R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted aromatic group, substituted benzyl group, Ring A when substituted and Ring B when further substituted, are substituted with one or more substituents selected from the group consisting of electron withdrawing group, halo, azido, —CN, —CONR⁸R⁹, —NR³R⁹, —OS(O)₂NR⁸R⁹, —S(O)₂NR⁸R⁹, —SO₃H, guanidino, oxalo, —C(═NR³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂), —NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
Q is —O—, —S—, —S(O)—, —S(O)₂—, —OS(O)₂—, —C(O)—, —OC(O)—, —C(O)O—, —C(O)C(O)—O—, —O—C(O)C(O)—, —NHC(O)—, —OC(O)NH—, —NH—C(O)—NH—, —S(O)₂NH—, —NHS(O)₂—, —C(NR¹⁴)NHNH—, —NHNHC(NR¹⁴)—, —NR⁸C(O)— or —NR⁸S(O)₂—;
R¹³ is a —H, —OH, —NH₂, an aromatic group or a substituted aromatic group;
R¹⁴ is —H, an aliphatic group, a benzyl group, an aryl group or non-aromatic heterocyclic group;
t is zero to three;
u is zero or one; and
p is one to five.

In some embodiments, n is one to four. In other embodiments n is one, two, or three. In preferred embodiments, n is two.

In some embodiments, R¹ is H. In other embodiments, R¹ is —OH. In preferred embodiments, R¹ is —OH.

In some embodiments, R² is an unsubstituted aromatic group. In other embodiments, R² is a substituted aromatic group. In other such embodiments, R² is phenyl. In other such embodiments, R² is a substituted phenyl group. In other such embodiments, R² is a substituted aromatic group, wherein said substituted aromatic group is 4-halophenyl selected from a group consisting of 4-chlorophenyl, 4-bromophenyl, and 4-fluorophenyl. In preferred embodiments, R² is 4-chlorophenyl.

In some embodiments, R³ and R⁴ are independently —H, an aliphatic group or a substituted aliphatic group. In some embodiments, R³ and R⁴ are both —H. In some embodiments, R³ and R⁴ are each independently an aliphatic group. In some embodiments, R³ and R⁴ are each independently a substituted aliphatic group. In some embodiments, R³ and R⁴ are each independently —H, or an aliphatic group. In some embodiments, R³ and R⁴ are each independently —H, or a substituted aliphatic group.

In some embodiments, R⁵ and R⁶ are each independently —H, an aliphatic group, or a substituted aliphatic group. In some embodiments, R⁵ and R⁶ are both —H. In some embodiments, R⁵ and R⁶ are each independently an aliphatic group. In some embodiments, R⁵ and R⁶ are each independently a substituted aliphatic group. In some embodiments, R⁵ and R⁶ are each independently —H, or an aliphatic group. In some embodiments; R¹ and R⁶ are each independently —H, or a substituted aliphatic group.

In some embodiments, at least one of R³, R⁴, R⁵ and R⁶ is an aliphatic group, or a substituted aliphatic group.

In some embodiments, at least one of R³, R⁴, R^{W} and R⁶ is an aliphatic group, or a substituted aliphatic group wherein:
said aliphatic group is a C₁-C₆ alkyl; and said substituted aliphatic group is a C₁-C₆ alkyl substituted with a substituent selected from the group consisting of —OH, —(O)ᵤ—(CH₂)ₜ—C(O)OR¹⁰, and —O-(aliphatic group);
t is zero to three;
u is zero or one; and
R¹⁰ is C₁-C₆ alkyl.

In some preferred embodiments, R³ and R⁴ are both —H; and R¹ and R⁶ are each independently selected from the group consisting of C₁-C₆ alkyl, and substituted C₁-C₆ alkyl.

In further preferred embodiments, R⁵ is CH₃. In other further preferred embodiments, both R⁵ and R⁶ are CH₃.

In some other preferred embodiments, R⁵ and R⁶ are both —H; and R³ and R⁴ are each independently selected from the group consisting of C₁-C₆ alkyl, and substituted C₁-C₆ alkyl.

In some further preferred embodiments, R³ is CH₃. In other further preferred embodiments, both R³ and R⁴ are CH₃.

In some embodiments Z is represented by formula (II):

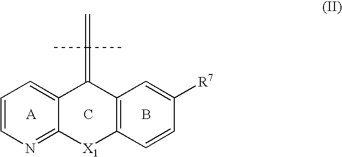

wherein:
ring A is unsubstituted or substituted;
ring B is further unsubstituted or substituted.

In some embodiments, rings A and B are both unsubstituted. In some embodiments, ring A is substituted as described above for an aromatic group, and ring B is further unsubstituted. In some other embodiments, ring B is further substituted as described above for an aromatic group, and ring A is unsubstituted. In some other embodiments, ring A is substituted and ring B is further substituted as described above for an aromatic group.

In some embodiments, R⁷ is —OH, —COOH, a halogen, —NO₂, an aliphatic group, a substituted aliphatic group, an aromatic group, a substituted aromatic group, —NR⁸R⁹, —CONR⁹R⁹, —C(═NR¹³)NR¹¹R¹², -Q-(aliphatic group), -Q-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², or —(O)ᵤ—(CH₂)ₜ—NHC(O)O—R¹⁰. Q, R⁸, R⁹, R¹⁰, R^{X}, R¹², R¹³, u and t are as described herein.

In some embodiments, R⁷ is an aliphatic group, a substituted aliphatic group, —O-(aliphatic group), or —O-(substituted aliphatic group). In other certain embodiments, R⁷ is an —O-alkyl, such as —O—CH₃, —O—C₂H₅, —O—C₃H₇, or —O—C₄H₉.

In another embodiment, R⁷ can be represented by —(O)ᵤ—(CH₂)ₜ—C(O)—NR¹¹R¹², wherein u is one, t is zero, and R¹¹ and R¹² are as described herein. In this embodiment, R¹¹ and R¹² can each independently be —H, a substituted or unsubstituted aliphatic group, a substituted or unsubstituted aromatic group, or R¹¹ and R¹² taken together with the nitrogen atom to which they are bonded form a substituted or unsubstituted nonaromatic heterocyclic ring (e.g., pyrrolidine, piperidine, morpholine).

In another embodiment, R⁷ can be represented by —(O)ᵤ—(CH₂)ₜ—C(O)—NR¹¹R¹², wherein u is zero, t is one to three, and R¹¹ and R¹² are as described herein.

In another embodiment, R⁷ can be represented by —(O)ᵤ—(CH₂)ₜ—C(O)—NR¹¹R¹², wherein both u and t are zero, and R¹¹ and R¹² are as described herein.

In another embodiment, R⁷ is an aliphatic group (e.g., methyl, ethyl, propyl) that is substituted with —NR⁸R⁹ or —CONR⁸R⁹, wherein R⁸ and R⁹ are as described herein. For example, R⁷ can be represented by:

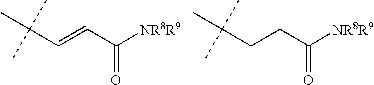

In another embodiment, R⁷ is —O—C(O)—NR¹¹R¹⁵, wherein R¹¹ is as described herein, R¹⁵ can be —H, an aliphatic group, a substituted aliphatic group, an aromatic group, a substituted aromatic group, a non-aromatic heterocyclic group, —C(O)—O-(substituted or unsubstituted aliphatic group), —C(O)—O-(substituted or unsubstituted aromatic group), —S(O)₂-(substituted or unsubstituted aliphatic group), —S(O)₂-(substituted or unsubstituted aromatic group) or R¹¹ and R¹⁵, taken together with the nitrogen atom to which they are bonded, can form a substituted or unsubstituted non-aromatic heterocyclic ring.

In other embodiments, R⁷ is a substituted aliphatic group, a substituted aromatic group, —O-substituted aliphatic group, or —O-substituted aromatic group. Preferably the aliphatic or aromatic moiety of the substituted aliphatic group, substituted aromatic group, —O-substituted aliphatic group or —O-substituted aromatic group bears a substituent selected from the group consisting of —OH, —COOR^{X}-Q-aliphatic group, or -Q-aromatic group. Q is as described herein. Preferably, Q is —C(O)O—. For example, R⁷ can be a linear, branched or cyclic aliphatic group that contains 1 to 6 carbon atoms, such as a C₁-C₆ alkyl group, a C₂-C₆ alkenyl, C₂-C₆ alkynyl, that is substituted with —OH, —COOH, —C(O)O—(C₁-C₆ aliphatic) or —C(O)O-(aromatic).

In additional embodiments, R⁷ can be —S(O)₂—NR¹¹R¹², or —N—C(O)—NR¹¹R¹², wherein R¹¹ and R¹² are as described herein.

In another preferred embodiment, the chemokine receptor antagonist can be represented by formula (I), wherein n is two, R¹ is —OH, R² is 4-halophenyl, and at least one of R³ and R⁴ is CH₃.

In another preferred embodiment, the chemokine receptor antagonist can be represented by formula (I), wherein n is two, R¹ is —OH, R² is 4-halophenyl, and both R³ and R⁴ are CH₃.

In a further preferred embodiment, the chemokine receptor antagonist can be represented by formula (I), wherein n is two, R¹ is —OH, R² is 4-chlorophenyl, and both R³ and R⁴ are independently CH₃.

In another embodiment, the method of the invention comprises administering a compound of formula (I):

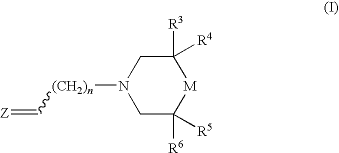

or a pharmaceutically acceptable salt or solvate thereof, or a pharmaceutical composition thereof, wherein:
n is one to four;
M is >CR¹R²;
R¹ is —OH;
R² is 4-halophenyl;
R³ and R⁴ are —H, and R⁵ and R⁶ are —CH—or
R³ and R⁴ are —CH₃ and R⁵ and R⁶ are —H;
Z is represented by formula (II):

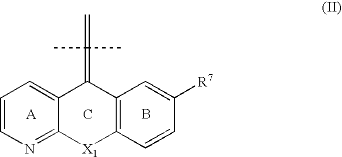

X₁ is —CH₂—O—; and
R⁷ is selected from the group consisting of:

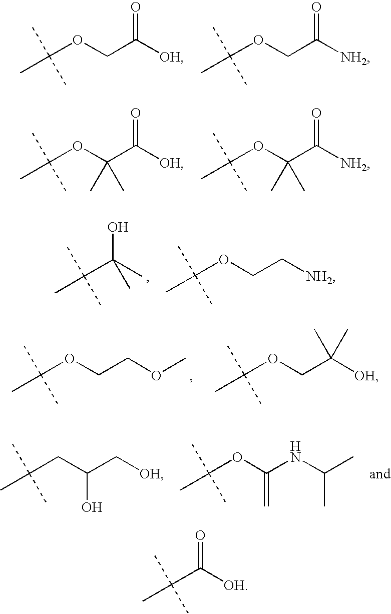

In some embodiments, for compounds described directly above R⁷ is

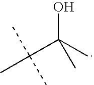

In some embodiments, for compounds described directly above, R⁷ is —COOH.

In some embodiments, the 4-halophenyl that is R² is selected from the group consisting of 4-chlorophenyl, 4-bromophenyl and 4-fluorophenyl. In preferred embodiments, the 4-halophenyl is 4-chlorophenyl.

In still other embodiments R³ and R⁴ are —H, R⁵ and R⁶ are —CH₃, n is two, and the compound is represented by formula (III):

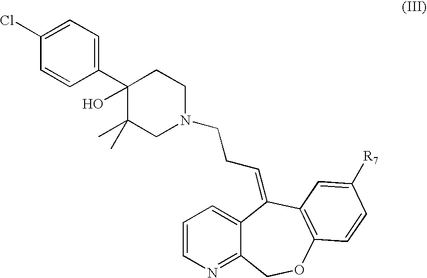

In another embodiment, the method of the invention comprises administering a compound of formula (IV):

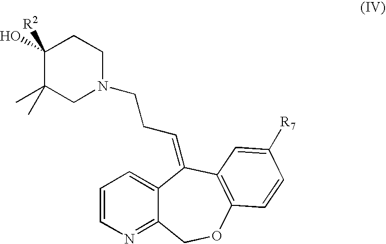

or a pharmaceutically acceptable salt or solvate thereof, or a pharmaceutical composition thereof wherein:
   R² is 4-halophenyl; and
   R⁷ is selected from the group consisting of:

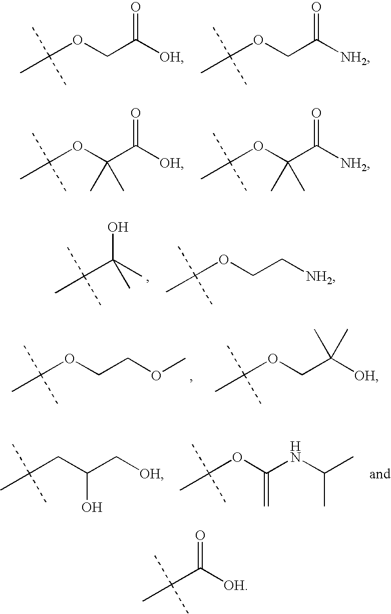

In some embodiments, for compounds described directly above, R⁷ is

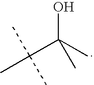

In some embodiments, for compounds described directly above, R⁷ is COOH.

In yet other embodiments, the 4-halophenyl is selected from the group consisting of 4-chlorophenyl, 4-bromophenyl, and 4-fluorophenyl. In preferred embodiments, the 4-halophenyl is 4-chlorophenyl.

Synthesis of Compounds for Use in the Method of the Invention

The compounds of this invention may be prepared in general by methods as illustrated in the following patents and publications: U.S. Pat. No. 6,613,905; U.S. Pat. No. 6,329,385; U.S. Pat. No. 6,509,346; WO01/09138; US2002/0169155; WO03/045942; WO04/043965; US 2004/0106639; WO 06/066200; and US 2007/0010545.

The entire contents of these patents and publications (and references cited therein) are hereby incorporated by reference. Exemplary compounds described in the foregoing publications and patents for use in the present invention include, but are not limited to the following:

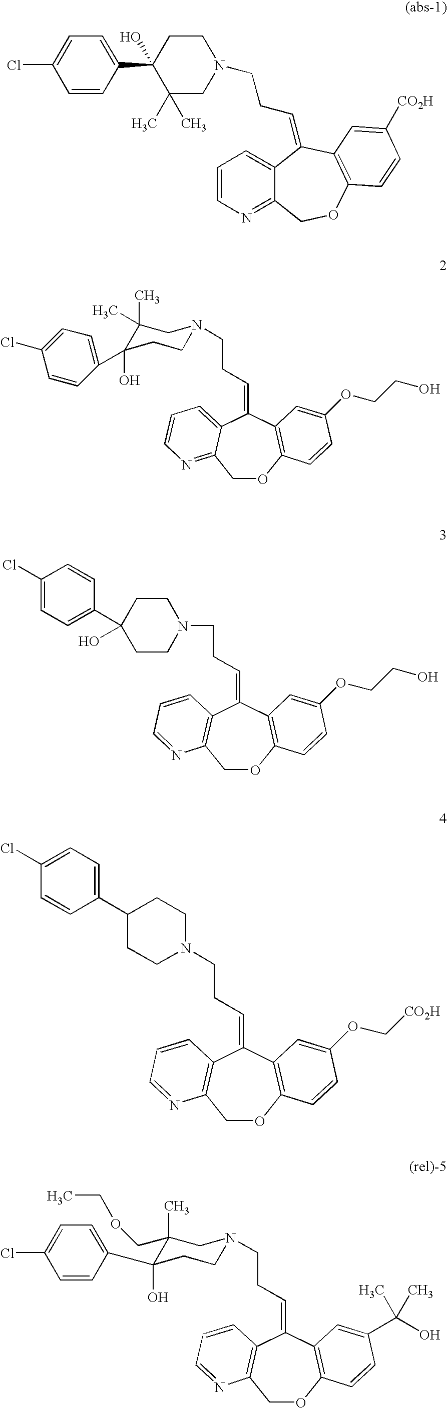

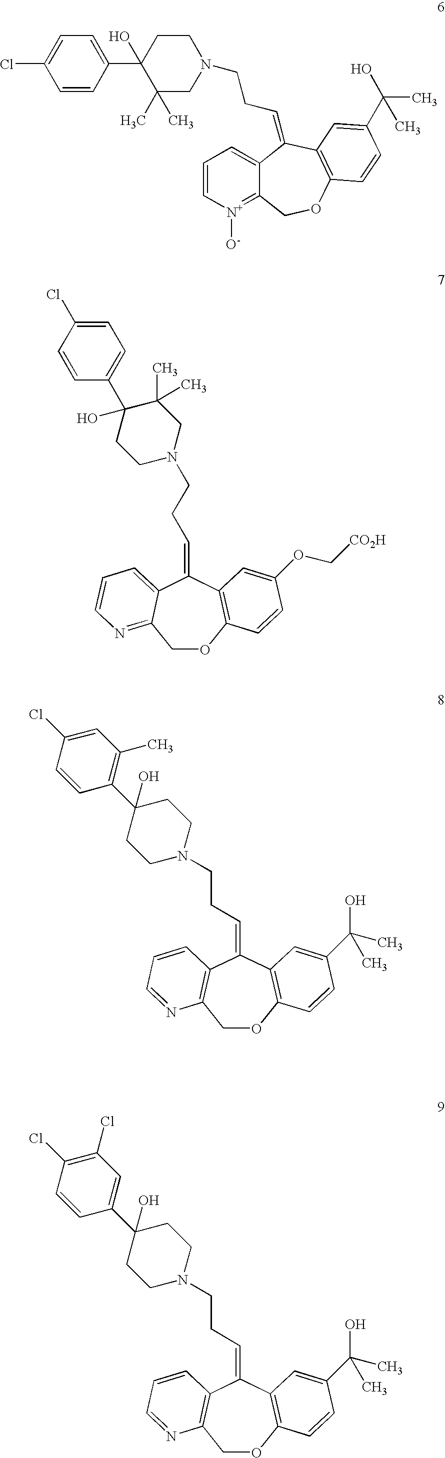

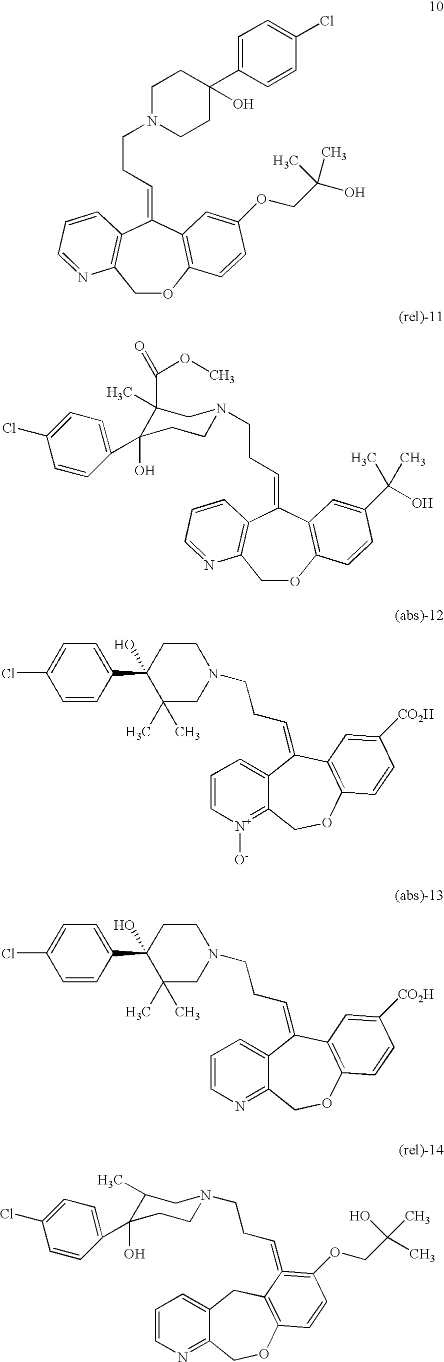

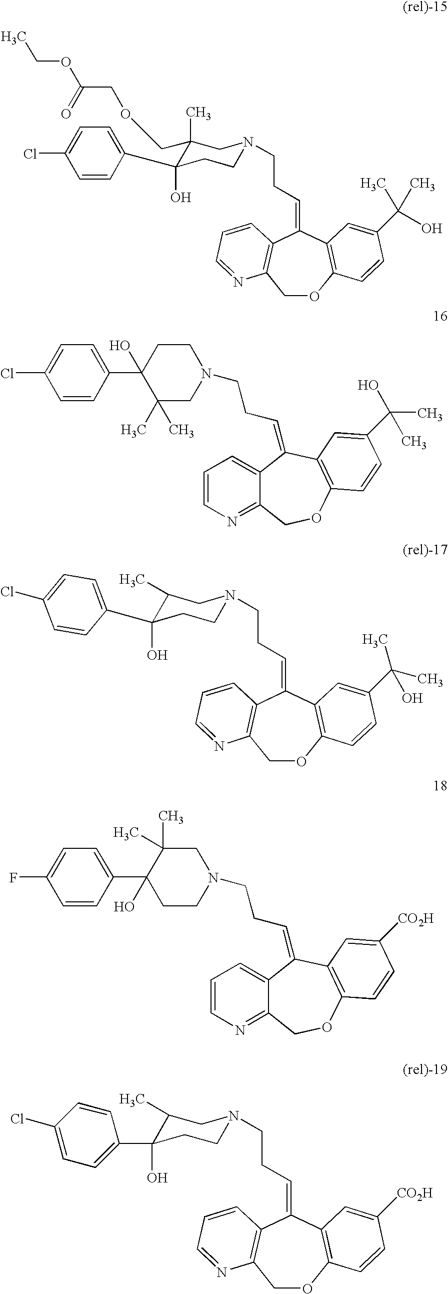

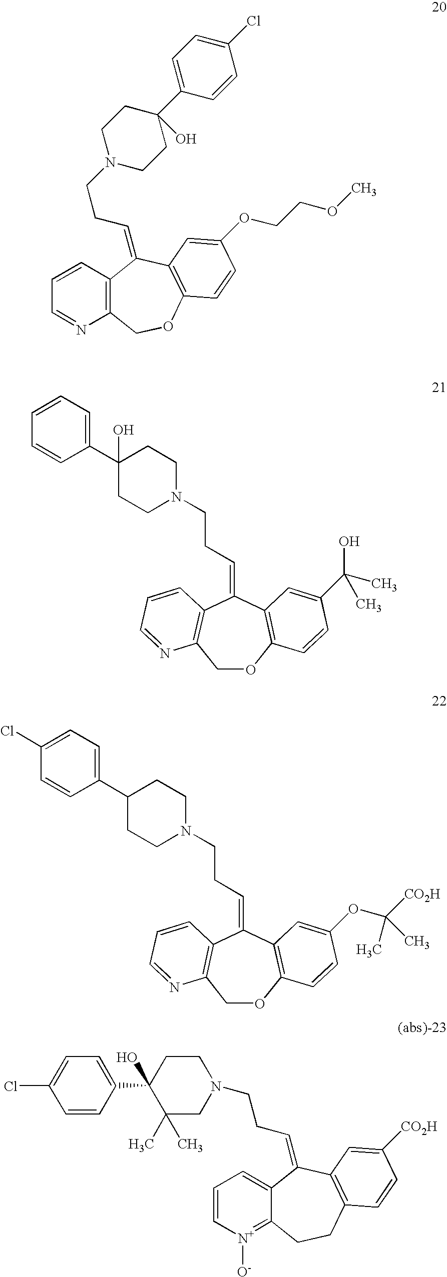

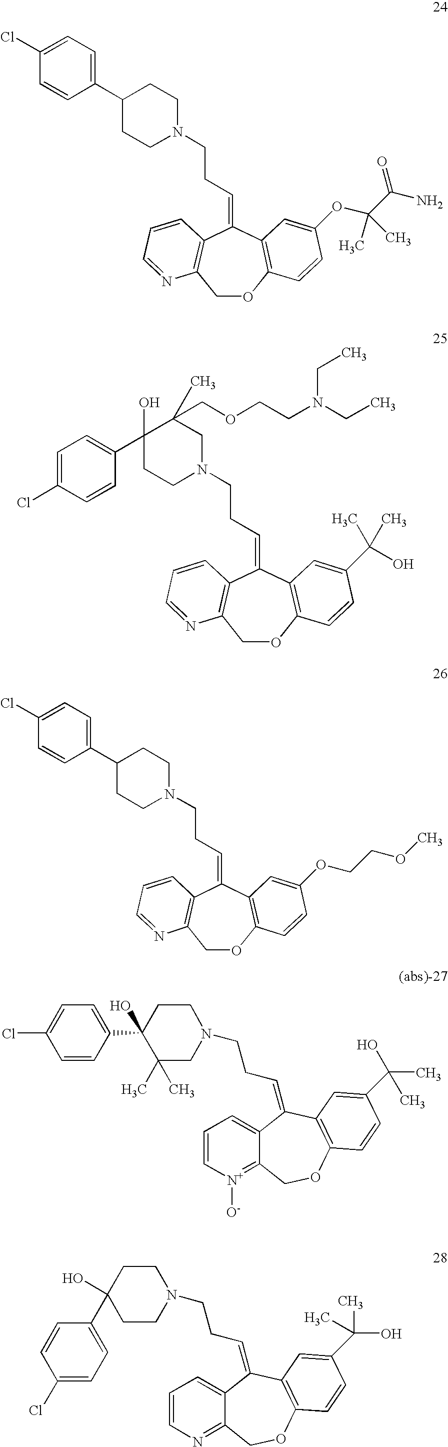

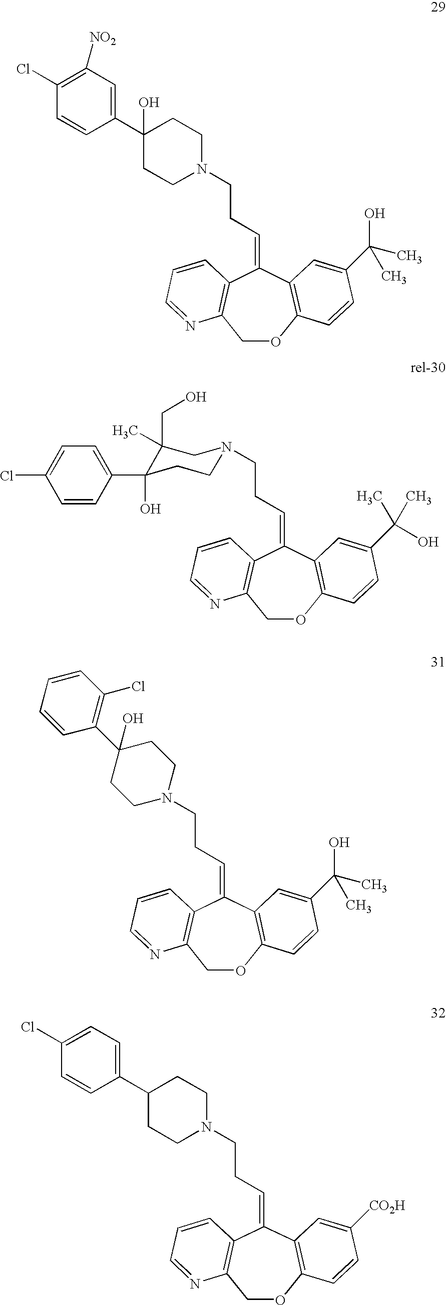

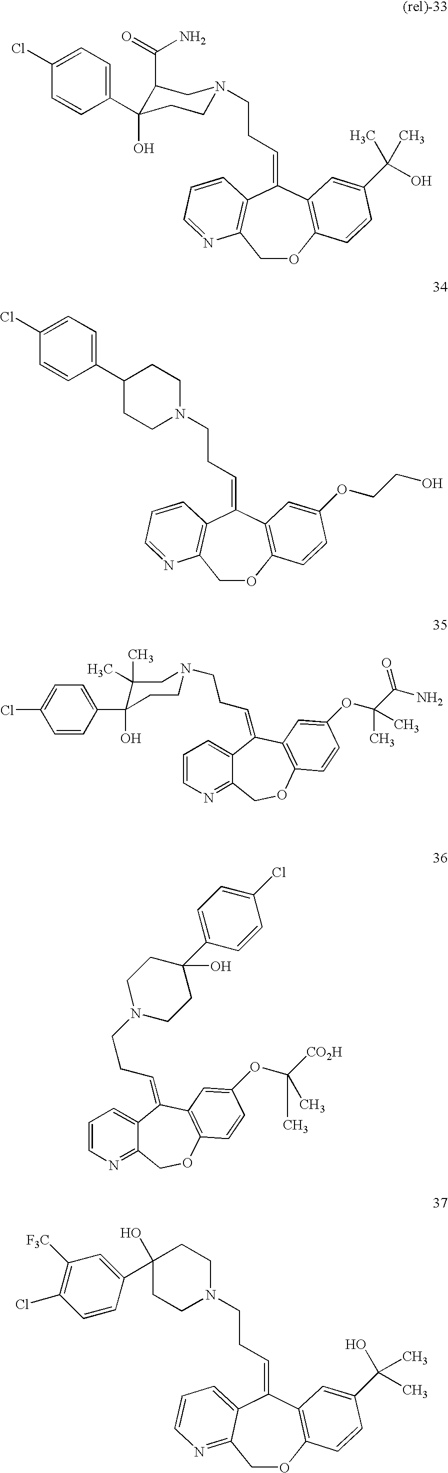

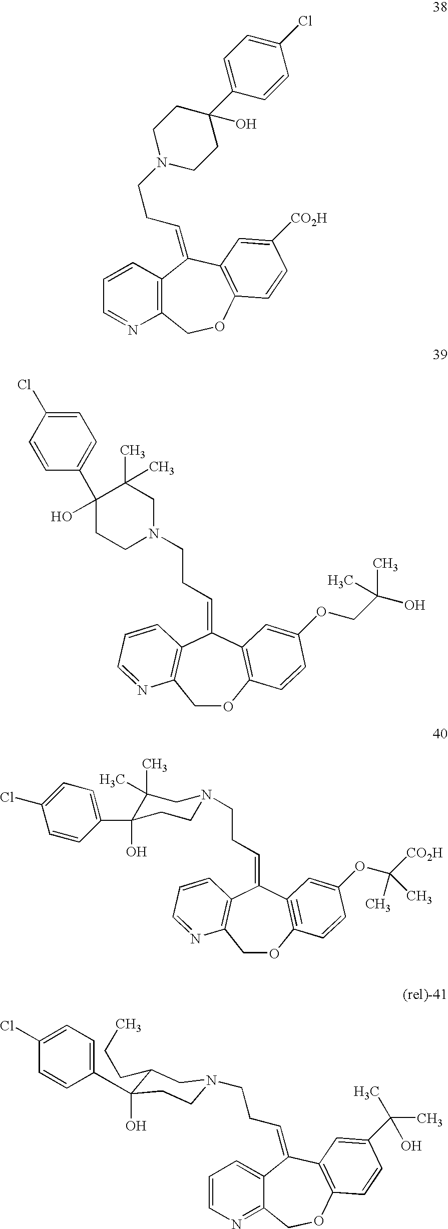

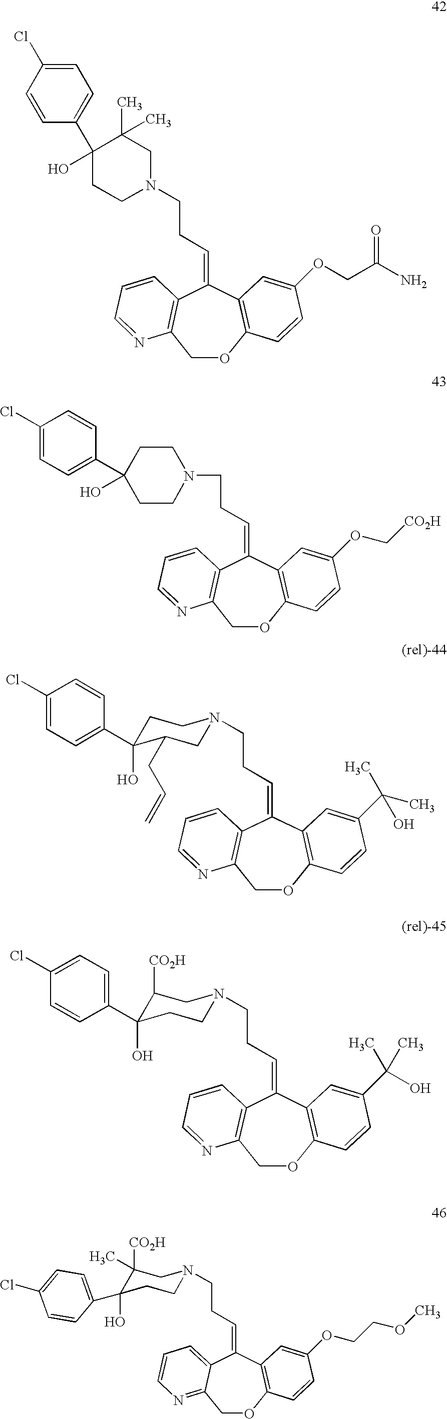

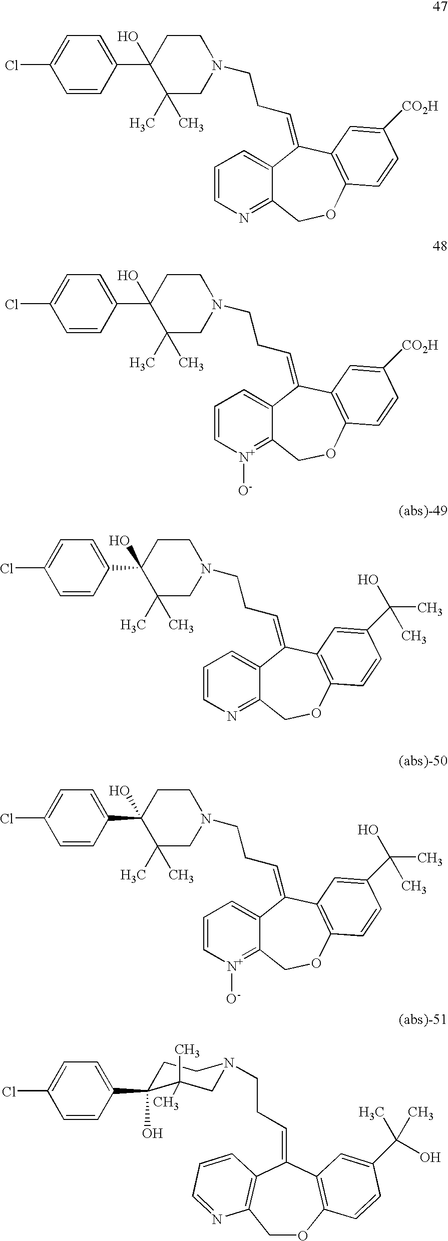

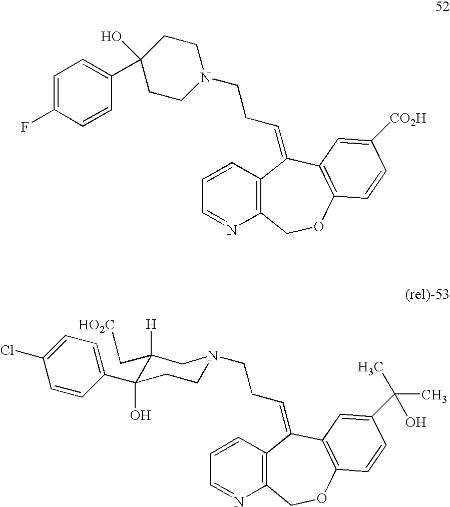

Pharmaceutical Compositions and Methods of Use

Compounds used in the methods of the invention can also be provided in the form of a pharmaceutical composition wherein these compositions comprise one or more of the compounds as described herein and a pharmaceutically acceptable carrier, adjuvant or vehicle. In certain embodiments, these compositions optionally further comprise one or more additional therapeutic agents.

Certain of the compounds of the present invention can exist in free form for treatment, or where appropriate, as a pharmaceutically acceptable salt or solvate thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt of a compound of this invention that, upon administration to a recipient, is capable of providing, either directly or indirectly, a compound of this invention or an active metabolite or residue thereof. As used herein, the term “active metabolite or residue thereof” means that a metabolite or residue thereof is useful for the treatment of inflammatory or allergic disorders. In some embodiments, without wishing to be bound by any particular theory, a “pharmaceutically acceptable salt” means any non-toxic salt of a compound of this invention that, upon administration to a recipient, is capable of providing, either directly or indirectly, an inhibitorily active compound of the invention or an inhibitorily active metabolite or residue thereof. As used herein, the term “inhibitorily active compound or inhibitorily active metabolite or residue thereof” means that a compound or metabolite or residue thereof is also an inhibitor of CCR1.

It will be appreciated that pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include but are not limited to adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

As described above, the pharmaceutical compositions additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, discloses various carriers used in formulating pharmaceutical compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutical composition, its use is contemplated to be within the scope of this invention.

As described above, compounds of the invention are useful for treating cancer and osteolytic bone disorders. In certain embodiments, compounds of the invention are useful for the treatment of multiple myeloma, both in its active form and during periods of clinical remission. In other certain embodiments, compounds of the invention are useful for the treatment of MGUS. In other certain embodiments, compounds of the invention are useful in the prevention, or delay, of the progression of MGUS to MM. In other certain embodiments, compounds of the invention are useful in the treatment of SMM. In other certain embodiments, compounds of the invention are useful in the prevention, or delay, of the progression of SMM to MM. In other certain embodiments, compounds of the invention are useful for the treatment of osteolytic bone disorders in multiple myeloma.

The term “osteolytic bone disorder” as used herein means a bone disorder in which bone resorption by osteoclasts exceeds bone production by osteoblasts, or in which it would be beneficial to reduce bone resorption by osteoclasts or chemotaxis of osteoclasts. Multiple myeloma is characterized by osteolytic bone lesions, and thus compounds of the invention are particularly useful for the treatment of multiple myeloma. Another example of an osteolytic bone disorder is Paget's disease, in which both osteoclasts and osteoblasts exhibit increased activity, yielding bone that is structurally unsound. Symptoms of Paget's disease include bone pain, bone deformity, and skeletal fragility. Other examples of disorders that may be treated using compounds of the invention include, but are not limited to secondary bone cancers, which originate in other parts of the body before spreading to the bone. In some embodiments, these cancers include, but are not limited to breast, lung, prostate, kidney and thyroid cancers.

As used herein, “treatment” or “treating” means partial alleviation, prevention, or cure of the disease.

As used herein an “effective amount” of the compound or pharmaceutical composition is that amount effective for treating a disease, condition, or disorder as described herein. The compounds and pharmaceutical compositions, according to the method of the present invention, may be administered using any amount and any route of administration effective for treating a disease, condition, or disorder as described herein. The skilled artisan will be able to determine appropriate dosages depending on these and other factors. An “effective amount” typically ranges between about 0.01 mg/kg/day to about 100 mg/kg/day, preferably between about 0.5 mg/kg/day to about 50 mg/kg/day. In other embodiments, an effective amount typically ranges between about 1 mg/kg/day to about 25 mg/kg/day.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The term “subject”, as used herein, is preferably a bird or mammal, such as a human (Homo sapiens), but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The pharmaceutical compositions of this invention can be administered to the subject orally, rectally, parenterally, intracisternally, intravaginally, intraperitoneally, topically (as by powders, ointments, or drops), bucally, as an oral or nasal spray, or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of this invention or pharmaceutical compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”. Exemplary additional therapeutic agents for use with an antagonist of chemokine receptor function include, but are not limited to theophylline, p-adrenergic bronchodilators, corticosteroids, antihistamines, antiallergic agents, immunosuppressive agents (e.g., cyclosporin A, FK-506, prednisone, methylprednisolone), hormones (e.g., adrenocorticotropic hormone (ACTH)), cytokines (e.g., interferons (e.g., IFNβ-1a, IFNβ-1β)), anticancer agents (particularly for the treatment of multiple myeloma), agents for the treatment of osteolytic bone disorders and the like.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In another aspect, the present invention provides a method for inhibiting cancer cell growth comprising contacting a cancer cell with an effective amount of one or more of the compounds as described herein.

For purposes of the invention, the term “cancer cell” refers to any cell that proliferates abnormally, including, without limitation, pancreatic, colon, breast, prostate, renal, lung, ovarian, gastric, esophageal, hepatocellular, or head and neck cancer cells, melanoma cells, leukemia cells, and multiple myeloma cells. In some embodiments, the cancer cell is grown in cell culture, including primary cultures and immortalized cell lines. In some other embodiments, the cancer cell is in an animal, preferably a mammal. As used herein, the term “mammal” includes, without limitation rats, mice, dogs, pigs, rabbits, non-human primates, and humans.

In another aspect, the present invention provides a method for inhibiting the adherence of a smoldering multiple myeloma cell to an osteoclast. In another aspect, the present invention provides a method for inhibiting the adherence of a multiple myeloma cell to an osteoclast. In another aspect, the present invention provides a method for reducing the secretion of growth factors from osteoclasts or surrounding stromal cells. In yet another aspect, the present invention provides a method for inhibiting osteoclast activity. In still another aspect, the present invention provides a method for inhibiting bone resorption resulting from increased osteoclast activity.

In order that this invention be more fully understood, the following preparative and testing examples are set forth.

### EXAMPLES

While the foregoing invention has been described in some detail for purposes of clarity and understanding, these particular embodiments are to be considered as illustrative and not restrictive. These examples illustrate how to make or test specific compounds, and are not to be construed as limiting the scope of the invention in any way. It will be appreciated by one skilled in the art from a reading of this disclosure that various changes in form and detail can be, made without departing from the true scope of the invention, which is to be defined by the appended claims rather than by the specific embodiments.

General Methods

Compounds of the invention have been shown to be inhibitors of CCR1. See U.S. Pat. No. 6,613,905; U.S. Pat. No. 6,329,385; U.S. Pat. No. 6,509,346; WO01/09138; US2002/0169155; WO03/045942; WO04/043965; US 2004/0106639; WO 06/066200; and US 2007/0010545. A general assay for determining the ability of compounds to inhibit CCR1 is described as follows (as also described in the foregoing applications and patents):

Membrane Preparations for Chemokine Binding and Binding Assays

Membranes were prepared from THP-1 cells (ATCC #TIB202). Cells were harvested by centrifugation, washed twice with PBS (phosphate-buffered saline), and the cell pellets were frozen at −70 to −85° C. The frozen pellet was thawed in ice-cold lysis buffer consisting of 5 mM HEPES (N-2-hydroxyethylpiperazine-N′-2-ethane-sulfonic acid) pH 7.5, 2 mM EDTA (ethylenediaminetetraacetic acid), 5 μg/ml each aprotinin, leupeptin, and chymostatin (protease inhibitors), and 100 μg/ml PMSF (phenyl methane sulfonyl fluoride—also a protease inhibitor), at a concentration of 1 to 5×10′ cells/ml. This procedure results in cell lysis. The suspension was mixed well to resuspend all of the frozen cell pellet. Nuclei and cell debris were removed by centrifugation of 400×g for 10 minutes at 4° C. The supernatant was transferred to a fresh tube and the membrane fragments were collected by centrifugation at 25,000×g for 30 minutes at 4° C. The supernatant was aspirated and the pellet was resuspended in freezing buffer consisting of 10 mM HEPES pH 7.5, 300 mM sucrose, 1 μg/ml each aprotinin, leupeptin, and chymostatin, and 10 μg/ml PMSF (approximately 0.1 ml per each 10⁸ cells). All clumps were resolved using a minihomogenizer, and the total protein concentration was determined using a protein assay kit (Bio-Rad, Hercules, Calif., cat #500-0002). The membrane solution was then aliquoted and frozen at −70 to −85° C. until needed.

Binding Assays utilized the membranes described above. Membrane protein (2 to 20 μg total membrane protein) was incubated with 0.1 to 0.2 nM ¹²⁵I-labeled RANTES or MIP-1α with or without unlabeled competitor (RANTES or MIP-1α) or various concentrations of compounds. The binding reactions were performed in 60 to 100 μl of a binding buffer consisting of 10 mM HEPES pH 7.2, 1 mM CaCl₂, 5 mM MgCl₂, and 0.5% BSA (bovine serum albumin), for 60 min at room temperature. The binding reactions were terminated by harvesting the membranes by rapid filtration through glass fiber filters (GF/B or GF/C, Packard) which were presoaked in 0.3% polyethyleneimine. The filters were rinsed with approximately 600 td of binding buffer containing 0.5 M NaCl, dried, and the amount of bound radioactivity was determined by scintillation counting in a Topcount beta-plate counter.

Other models include those referenced herein and also include:

a. Oncogene 20: 4519-4527 (2001); and

b. Blood 103: 3474-3479 (2004); and

c. Blood 106: 713-716 (2005).

Other General Methods

Compound 51 ((4S)-4-(4-chlorophenyl)-1-13-[7-(1-hydroxy-1-methylethyl)[1]benzoxepino[3,4-b]pyridin-5(11H)-ylidene]propyl)-3,3-dimethylpiperidin-4-ol) was used in Examples 1-5. Compound 51 was dissolved in dimethyl sulfoxide (DMSO; Sigma Chemical, St Louis, Mo.) at 10 mM, and stored at −20° C. until use. For each experiment, it was diluted immediately before use in culture medium (0.2-100 nM) with less than 0.002% of DMSO.

Cell lines: The dexamethasone (Dex)-sensitive (MM.1S) human MM celline was provided by Dr Steven Rosen (Northwestern University, Chicago, Ill.). The INA6 human IL-6-dependent MM cell line was provided by Dr Renate Burger (University of Kiel, Kiel, Germany) (see Burger et al., J. Hematol. 2:42-53 (2001)) and cultured in the presence of 2.5 ng/mL IL-6 (R&D Systems, Minneapolis, Minn.). The OPM1 myeloma cell line was provided by Dr Lief Bergsagel (Mayo Clinic, Scottsdale, Ariz.), and the U266 cell line was obtained from the American Type Culture Collection (Rockville, Md.). All MM cell lines were cultured in RPMI 1640 media (Sigma Chemical) containing 10% fetal bovine serum (FBS), 2 mM L-glutamine, 100 U/mL penicillin, and 100 μg/mL streptomycin (Gibco, Grand Island, N.Y.).

MM primary cells: Patient tumor cells were isolated as described in Kiziltepe et al., Blood 110:709-718 (2007). Following appropriate informed consent, obtained in accordance with the Declaration of Helsinki and with approval by the Institutional Review Board of the Dana-Farber Cancer Institute (Boston, Mass.), MM patient cells were separated from bone marrow (BM) samples by antibody-mediated positive selection using anti-CD138 magnetic activated cell separation microbeads (Miltenyi Biotech, Gladbach, Germany).

Osteoclast formation. Osteoclasts were generated from peripheral blood mononuclear cells (PBMCs) from healthy volunteers by Ficoll-Paque gradient separation and cultured in 6-well or 96-well plates (0.5×10⁶ cells/cm²). After 2 hours, nonadherent PBMCs were removed, and adherent cells were cultured for 21 days in e-MEM containing 10% FBS and 1% penicillin-streptomycin (Mediatech, Herndon, Va.), as well as 50 ng/mL of macrophage colony-stimulating factor (M-CSF; R&D Systems, Minneapolis, Minn.) and RANKL (PeproTech, Rocky Hill, N.J.).

Co-culture experiments: Osteoclasts were harvested with cell dissociation buffer (Invitrogen, Carlsbad, Calif.) and seeded in 96-well or 24-well plates (approximately 1.5-3×10⁴ cells/cm²). After washing, multiple myeloma cells were added to the wells and incubated with media or with Compound 51 (10 nM) for the specified times at 37° C. For multiple myeloma cell proliferation, DNA synthesis was measured by tritiated thymidine uptake (3H-TdR; Perkin Elmer, Boston, Mass.), pulsing multiple myeloma cells with 3H-TdR (0.5 μCi/well [0.0185 MBq]L) during the last 8 hours of 48-hour cultures. At the end of the culture, cells were harvested onto paper filters with an automatic cell harvester (Cambridge Technology, Cambridge, Mass.) and counted using the LKB Betaplate scintillation counter (Wallac, Gaithersburg, Md.). To assess cell survival, viable multiple myeloma cells were counted by trypan blue staining.

CCR1 Inhibition Blocks Osteoclast Formation and Activity

### Example 1

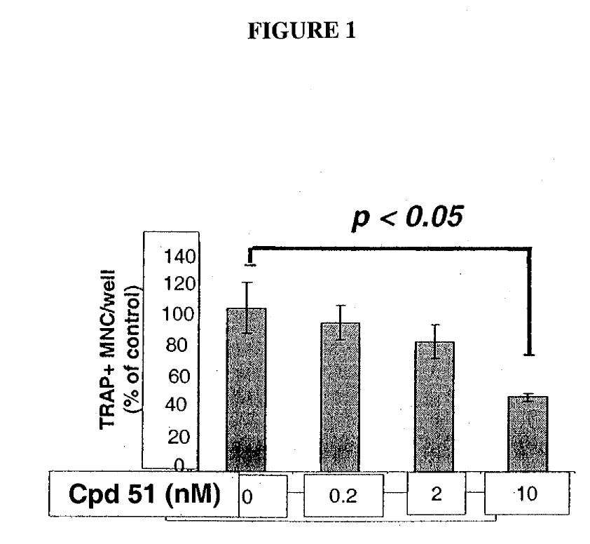

Compound 51 was added to osteoclast cultures at concentrations and time points as shown in FIG. 1, and culture media was replaced twice weekly. After 3 weeks, cells were fixed with citrate-acetone solution and stained for tartrate-resistant acid phosphatase (TRAP) using an acid phosphatase leukocyte staining kit (Sigma Chemical) according to the manufacturer's instructions. TRAP′ OCs containing 3 or more nuclei per cell were enumerated. Each OC formation assay was performed at least 3 times using PBMCs from different donors. As shown in FIG. 1, Compound 51 (10 nM) reduced osteoclast number by 40-60% compared to control (p<0.05).

No dose-dependent effect was seen as doses up to 100 nM did not further decrease osteoclast number, therefore all subsequent experiments used 10 nM Compound 51 unless otherwise stated.

### Example 2

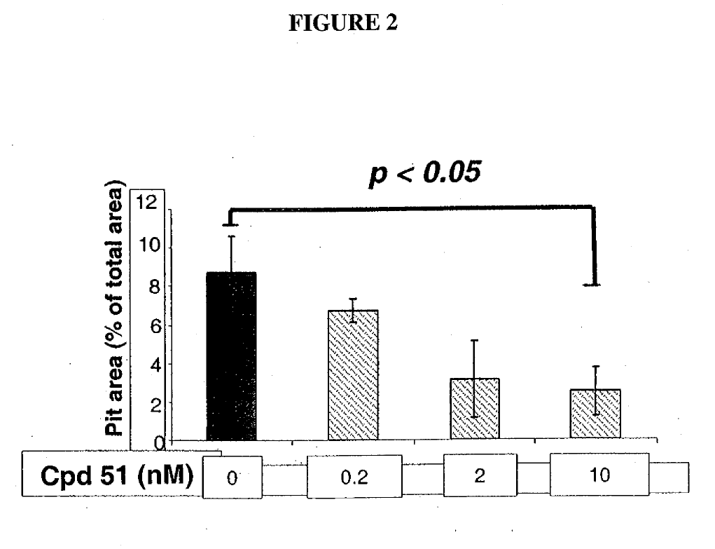

Pit formation assay. Osteoclast activity was assayed by bone resorption enumerating resorption pits. PBMCs were cultured (0.5×10⁶ cells/well) on dentin slices (Immunodiagnostic Systems, Boldon, United Kingdom) in 96-well plates as per the manufacturer's guidelines, and then stimulated with RANKL and M-CSF (50 ng/mL); Compound 51 was added as indicated in FIG. 2. After 3 weeks, adherent cells were scraped off gently with 0.1% Triton. Bone slices were washed in distilled water and stained with 1% toluidine solution. Resorption pits were then quantified by light microscopy using the public domain National Institutes of Health (NIH) Image J software version 1.36b. Each pit area assay was performed at least 3 times with PBMCs from different donors. As shown in FIG. 2, almost complete abrogation of the characteristic resorptive tracks and a reduction of pit numbers was seen in the presence of Compound 51 (10 nM). Consistent with the reduction of osteoclast formation, Compound 51 (10 nM) significantly reduced bone resorption areas (mean±SD, 2.4%±1.2% vs 8.7%±1.9% of total area per slice in the control; P<0.01).

### CCR1 Inhibition Blocks Adhesion Between MM Cells and Osteoclasts

### Example 3

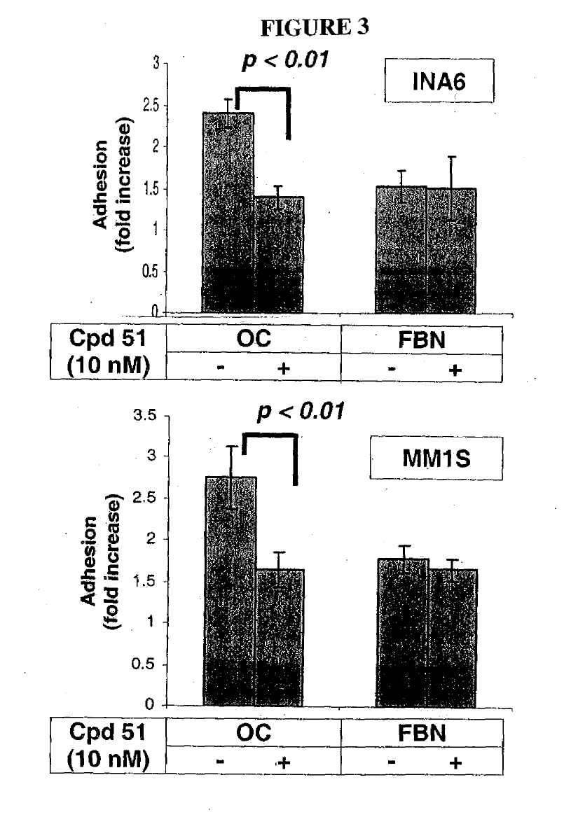

Adhesion Assay. Multiple myeloma cell lines were labeled with calcein AM (Invitrogen) according to the manufacturer's instructions and plated in a 96-well plates with OCs, fibronectin (FBN; 20 μg/mL), or media, with or without Compound 51. After 6 hours of incubation, plates were washed and fluorescence of the adherent cells was measured using the Multimode Reader Mithras LB 940 (Berthold Technologies, Wildbad, Germany). As shown in FIG. 3, multiple myeloma cell adhesion to osteoclasts was almost completely inhibited by Compound 51 and this was independent of CCR1 expression on MM cells. As also shown in FIG. 3, there were no effects on MM cell adhesion to fibronection (FBN).

### CCR1 Inhibition Abrogates MM Cell Survival and Proliferative Advantage Conferred by Osteoclasts

### Example 4

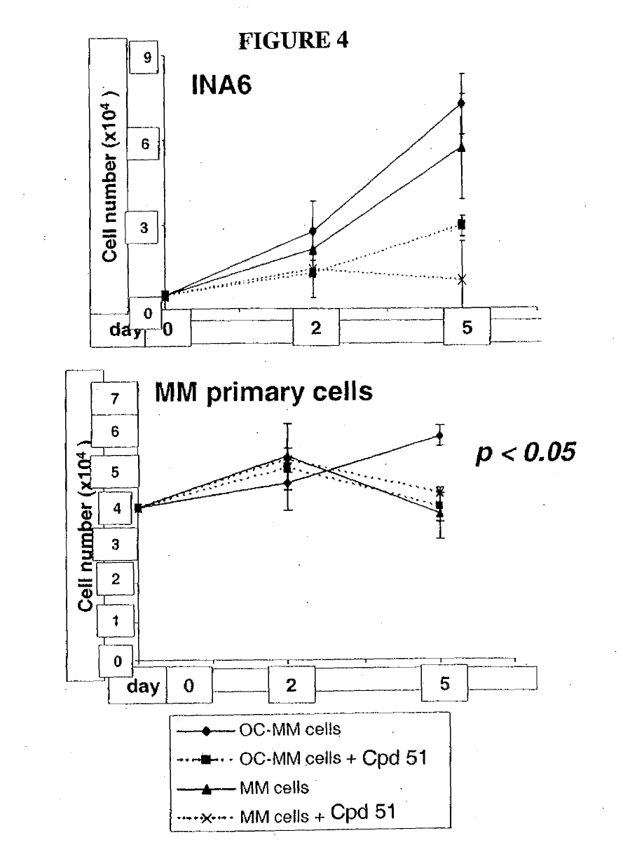

Long-term co-culture. Osteoclasts were plated in a 24-well plate (3×10⁴ cells/well) with either INA6 multiple myeloma cells (5×10³ cells/well) or primary patient multiple myeloma cells (3×10⁵ cells/well) for 5 days using the general method described above. As shown in FIG. 4, osteoclasts promote proliferation and survival of multiple myeloma cells, consistent with a previous report (Abe et al., Blood 104:2484-2491 (2004)). As further shown in FIG. 4, Compound 51 almost completely abrogated this survival advantage in both INA6 and primary MM cells. This same effect was also noted in MM1.S cells (data not shown).

### Example 5

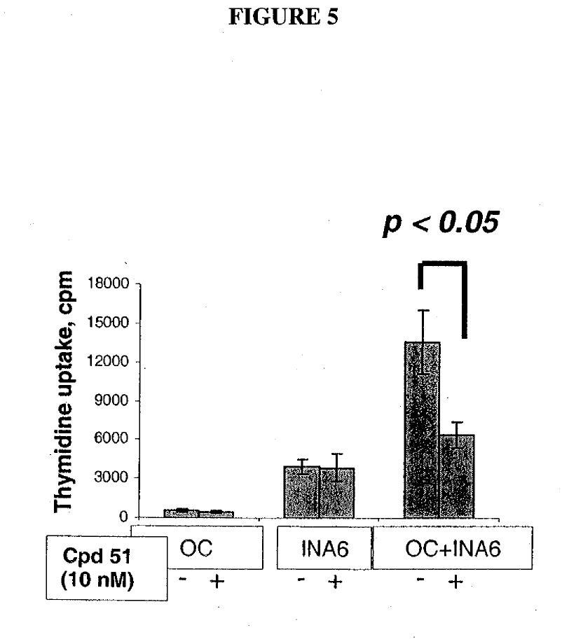

Short-term co-culture. Osteoclasts were seeded in a 96-well plate at a density of 10⁴ cells/well and cocultured with INA6 cells (3×10⁴ cells/well) for 48 hours. As shown in FIG. 5, osteoclasts stimulate multiple myeloma cell proliferation, as assessed at 48 hours by thymidine uptake, in INA6 cells (3.5-fold increase over that of control; and other cell lines (data not shown). Compound 51 showed only modest antiproliferative effects on MM cells alone; however, it blocked induction of proliferation by OCs, reducing MM cell proliferative response by 50% (P<0.05).

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

What is claimed is:

1. A method of treating multiple myeloma, smoldering multiple myeloma, a secondary bone cancer or an osteolytic bone disorder comprising administering to a subject a compound of formula (I): 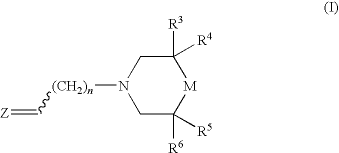
or a pharmaceutically acceptable salt or solvate thereof, or a pharmaceutical composition thereof, wherein:
n is one to four;
M is >CR¹R²;
R¹ is OH or H;
R² is a substituted or unsubstituted aromatic group;
R³ and R⁴ are independently —H, an aliphatic group or a substituted aliphatic group;
R¹ and R⁶ are independently —H, an aliphatic group or a substituted aliphatic group;
Z is represented by formula (II): 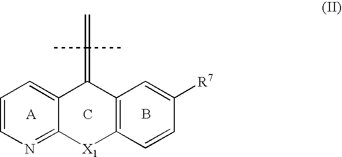
wherein:
ring A is unsubstituted or substituted;
ring B is further unsubstituted or substituted;
R⁷ is —OH, —COOH, —NO₂, halogen, aliphatic group, substituted aliphatic group, an aromatic group, a substituted aromatic group, —NR⁸R⁹, —CONR⁸R⁹, —NR⁸C(O)-(aliphatic group), —NR⁸C(O)-(substituted aliphatic group), —NR⁸S(O)₂-(aliphatic group), —NR⁸S(O)₂-(substituted aliphatic group), —C(O)O-(aliphatic group), —C(O)O-(substituted aliphatic group), —C(O)-(aliphatic group), —C(O)-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR²⁰, —(O)ᵤ—(CH₂)ₜ—OC(O)R¹⁰, —(O)ᵤ—(CH₂)ₜ—C(O)—NR¹¹R¹² or —(O)ᵤ—(CH₂)ₜ—NHC(O)O—R¹⁰;
R⁸ and R⁹ are independently —H, an aliphatic group or a substituted aliphatic group, a benzyl group, an aromatic group, non-aromatic heterocyclic group; or R⁸ and R⁹ taken together with the nitrogen atom to which they are bonded can form a substituted or unsubstituted non-aromatic heterocyclic ring;
R¹⁰, R¹¹ or R¹² are independently —H, an aliphatic group, a substituted aliphatic group, an aromatic group, a substituted aromatic group or a non-aromatic heterocyclic group, —NHC(O)—O-(aliphatic group), —NHC(O)—O-(aromatic group) or —NHC(O)—O-(non-aromatic heterocyclic group); or R¹¹ and R¹², taken together with the nitrogen atom to which they are bonded, form a non-aromatic heterocyclic ring;
X₁ is —CH₂—O—;
said aliphatic group is a C₁-C₆ alkyl, alkenyl or alkynyl;
said aromatic group is selected from the group consisting of phenyl, 1-naphthyl, 2-naphthyl, 1-anthracyl, 2-anthracyl, N-imidazolyl, 2-imidazolyl, 4-imidazolyl, 5-imidazolyl, 2-thienyl, 3-thienyl, 2-furanyl, 3-furanyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 4-pyridazinyl, 3-pyrazolyl, 4-pyrazolyl, 5-pyrazolyl, 2-pyrazinyl, 2-thiazolyl, 4-thiazolyl, 5-thiazolyl, 5-tetrazolyl, 2-oxazolyl, 4-oxazolyl, 5-oxazolyl, tetrahydronaphthyl, 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 1-isoquinolinyl, 3-quinolinyl, 1-isoindolyl, 3-isoindolyl, acridinyl, 3-benzisoxazolyl, benzocyclopentyl, benzocyclohexyl;
said non-aromatic heterocyclic group is a five to eight-membered non-aromatic ring which contains one or more heteroatoms independently selected from the group consisting of nitrogen, oxygen or sulfur;
said substituted aliphatic group is substituted with one or more substituents selected from the group consisting of oxo group, epoxy group, non-aromatic heterocyclic ring, benzyl group, substituted benzyl group, aromatic group, substituted aromatic group, electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂), —NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted non-aromatic heterocyclic group is substituted with one or more substituents selected from the group consisting of ═O, ═S, electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR¹³)NR¹¹R¹², ═NR¹³, —(O)ᵤ—(CH₂)ₜ—C(O)OR¹⁰, —(O)ᵤ—(CH₂)ₜ—OC(O)R¹⁰, —(O)ᵤ—(CH₂)ₜ—C(O)—NR¹¹R¹², —(O)ᵤ—(CH₂)ₜ—NHC(O)O—R¹⁰, —H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
said substituted aromatic group, substituted benzyl group, Ring A when substituted and Ring B when further substituted, are substituted with one or more substituents selected from the group consisting of electron withdrawing group, halo, azido, —CN, —CONR⁸R⁹, —NR⁸R⁹, —OS(O)₂NR⁸R⁹, —S(O)₂NR⁸R⁹, —SO₃H, guanidino, oxalo, —C(═NR³)NR¹¹R¹², ═NR¹³, —(O)ᵤ—(CH₂)ₜ—C(O)OR¹⁰, —(O)ᵤ—(CH₂)ₜ—OC(O)R¹¹, —(O)ᵤ—(CH₂)ₜ—C(O)—NR¹¹R¹², —(O)ᵤ—(CH₂)ₜ—NHC(O)O—R¹⁰, -Q-H, -Q-(aliphatic group), -Q-(substituted aliphatic group), -Q-(aryl), -Q-(aromatic group), -Q-(substituted aromatic group), -Q-(CH₂)ₚ-(substituted or unsubstituted aromatic group), -Q-(non-aromatic heterocyclic group) or -Q-(CH₂)ₚ-(non-aromatic heterocyclic group);
Q is —O—, —S—, —S(O)—, —S(O)₂—, —OS(O)₂—, —C(O)—, —OC(O)—, —C(O)O—, —C(O)C(O)—O—, —O—C(O)C(O)—, —NHC(O)—, —OC(O)NH—, —NH—C(O)—NH—, —S(O)₂NH—, —NHS(O)₂—, —C(NR¹⁴)NHNH—, —NHNHC(NR¹⁴)—, —NR⁸C(O)— or —NR⁸S(O)₂—;
R¹³ is a —H, —OH, —NH₂, an aromatic group or a substituted aromatic group;
R¹⁴ is —H, an aliphatic group, a benzyl group, an aryl group or non-aromatic heterocyclic group;
t is zero to three;
u is zero or one; and
p is one to five.

2. The method of claim 1, wherein the method is the treatment of multiple myeloma.

3. The method of claim 1, wherein the method is the treatment of smoldering multiple myeloma.

4. The method of claim 1, wherein:
R² is a substituted aromatic group; and
said substituted aromatic group is 4-halophenyl, selected from the group consisting of 4-chlorophenyl, 4-bromophenyl and 4-fluorophenyl.

5. The method of claim 4, wherein said 4-halophenyl is 4-chlorophenyl.

6. The method of claim 1, wherein:
at least one of R³, R⁴, R⁵ and R⁶ is an aliphatic group or a substituted aliphatic group;
said aliphatic group is a C₁-C₆ alkyl and said substituted aliphatic group is a C₁-C₆ alkyl substituted with a substituent selected from the group consisting of —OH, —(O)ᵤ—(CH₂), —C(O)OR¹⁰ and —O-(aliphatic group);
t is zero to three;
u is zero or one; and
R¹⁰ is C₁-C₆ alkyl.

7. The method of claim 6, wherein:
R³ and R⁴ are both —H; and
R⁵ and R⁶ are independently selected from the group consisting of C₁-C₆ alkyl and substituted C₁-C₆ alkyl.

8. The method of claim 7, wherein R⁵ is —CH₃.

9. A method of treating multiple myeloma, smoldering multiple myeloma, a secondary bone cancer or an osteolytic bone disorder comprising administering to a subject a compound of formula (I): 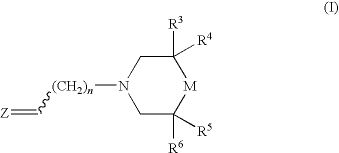
or a pharmaceutically acceptable salt or solvate thereof, or a pharmaceutical composition thereof, wherein:
n is one to four;
M is >CR¹R²;
R¹ is —OH;
R² is 4-halophenyl;
R³ and R⁴ are —H, and R¹ and R⁶ are —CH—or
R³ and R⁴ are —CH₃ and R⁵ and R⁶ are —H;
Z is represented by formula (II): 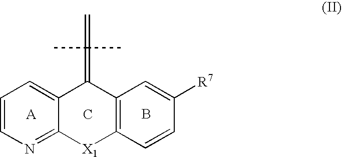
X₁ is —CH₂—O—; and
R⁷ is selected from the group consisting of:

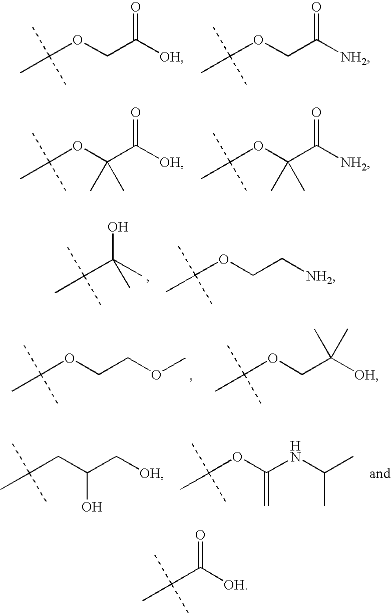

10. The method of claim 9, wherein the method is the treatment of multiple myeloma.

11. The method of claim 9, wherein the method is the treatment of smoldering multiple myeloma.

12. The method of claim 9, wherein R⁷ is:

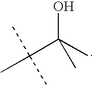

13. The method of claim 9, wherein R⁷ is —COOH.

14. The method of claim 9, wherein said 4-halophenyl is selected from the group consisting of 4-chlorophenyl, 4-bromophenyl and 4-fluorophenyl.

15. The method of claim 14, wherein said 4-halophenyl is 4-chlorophenyl.

16. The method of claim 15, wherein:
R³ and R⁴ are —H, R⁵ and R⁶ are —CH₃, n is two, and the compound has the structure of formula (III):

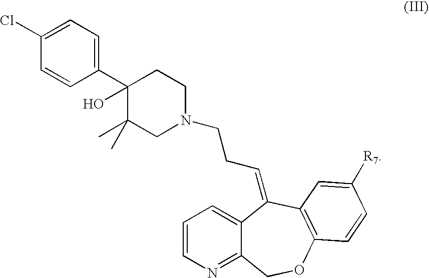

17. A method of treating multiple myeloma, smoldering multiple myeloma, a secondary bone cancer or an osteolytic bone disorder comprising administering comprising administering to a subject a compound of formula (IV): 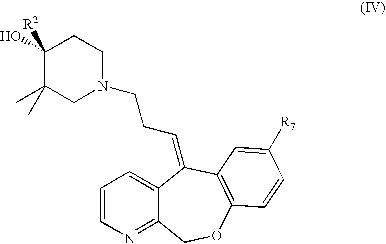
or a pharmaceutically acceptable salt or solvate thereof, or a pharmaceutical composition thereof wherein:
R² is 4-halophenyl; and
R⁷ is selected from the group consisting of:

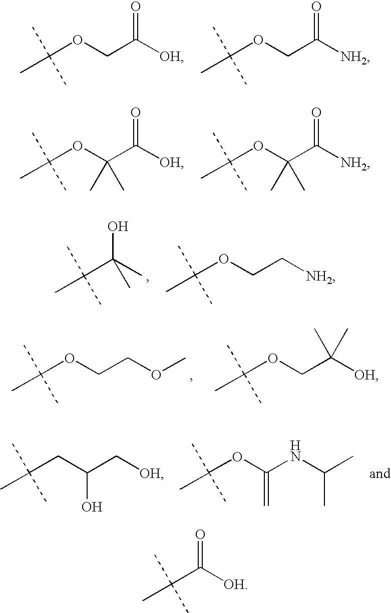

18. The method of claim 17, wherein the method is the treatment of multiple myeloma.

19. The method of claim 17, wherein the method is the treatment of smoldering multiple myeloma.

20. The method of claim 17, wherein R⁷ is

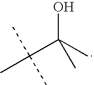

21. The method of claim 17, wherein R⁷ is COOH.

22. The method of claim 17, wherein R² is selected from the group consisting of 4-chlorophenyl, 4-bromophenyl and 4-fluorophenyl.

23. The method of claim 22, wherein R² is 4-chlorophenyl.
